(19) United States
(12) Patent Application Publication
(10) Pub. No.: US 2023/0340571 A1
(43) Pub. Date: Oct. 26, 2023

(54) MACHINE-LEARNING MODELS FOR SELECTING OLIGONUCLEOTIDE PROBES FOR ARRAY TECHNOLOGIES

(71) Applicants: Illumina, Inc., San Diego, CA (US); Illumina Software, Inc., San Diego, CA (US)

(72) Inventors: Sepideh Almasi, Los Gatos, CA (US); Yong Li, San Diego, CA (US); Anindita Dutta, San Francisco, CA (US); Eric Vermaas, San Diego, CA (US); Rigoberto Pantoja, San Diego, CA (US)

(21) Appl. No.: 18/307,482

(22) Filed: Apr. 26, 2023

Related U.S. Application Data
(60) Provisional application No. 63/363,618, filed on Apr. 26, 2022.

(51) Int. Cl. C12Q 1/6811; C12Q 1/6874; G16B 25/20

(52) U.S. Cl. CPC C12Q 1/6811; C12Q 1/6874; G16B 25/20; C12Q 2600/156

(57) ABSTRACT

This disclosure describes methods, non-transitory computer readable media, and systems that can use a machine-learning model to classify or predict a probability of an oligonucleotide probe yielding an accurate genotype call or hybridizing with a target oligonucleotide—based on the oligonucleotide probe's nucleotide-sequence composition. To intelligently identify oligonucleotide probes that are more likely to yield accurate downstream genotyping—or more likely to successfully hybridize with target oligonucleotides—some embodiments of the disclosed machine-learning model include customized layers trained to detect motifs or other nucleotide-sequence patterns that correlate with favorable or unfavorable probe accuracy. By intelligently processing the nucleotide sequences of candidate oligonucleotide probes before implementing a microarray for a particular target oligonucleotide, the disclosed system can identify oligonucleotide probes with better genotyping accuracy (or better binding accuracy) than existing microarray systems for use in a microarray.

### CROSS-REFERENCE TO RELATED APPLICATIONS

The present application claims the benefit of, and priority to, U.S. Provisional Application No. 63/363,618, entitled “MACHINE-LEARNING MODELS FOR SELECTING OLIGONUCLEOTIDE PROBES FOR ARRAY TECHNOLOGIES,” filed on Apr. 26, 2022. The aforementioned application is hereby incorporated by reference in its entirety.

### BACKGROUND

In recent years, biotechnology firms and research institutions have improved hardware and software for microarrays that determine genotypes of targeted nucleotide sequences within a genomic sample. For instance, existing microarray systems can use oligonucleotide probes to hybridize with respective target oligonucleotides from a genomic sample and determine genotypes for respective target oligonucleotides upon detecting (or not detecting) such hybridization. In some cases, existing microarray systems attach or embed copies of a deoxyribonucleic acid (DNA) probe to a slide or chip (e.g., a bead on a flow cell or other slide), where the DNA probe includes a fluorescent tag or other label that can be added as nucleobases are incorporated to extend the DNA probe; introduce, to the slide or chip, a solution comprising a genomic sample's oligonucleotide fragments; and (after washing the slide or chip) scan the surface with a camera to detect whether the oligonucleotide fragments hybridize with the DNA probe and extend the probe with fluorescently labelled nucleobases. When the scan detects a light emitted by the labeled DNA probes, an existing microarray system (i) determines that a target oligonucleotide corresponding to the DNA probe is present in the genomic sample and (ii) generates a corresponding genotype call for the target oligonucleotide. For instance, existing microarray systems can generate genotype calls representing single nucleotide polymorphisms (SNPs), insertions or deletions (indels), or other variants corresponding to the DNA probe.

Despite recent advances in designing oligonucleotide probes for microarrays, existing microarray systems frequently use oligonucleotide probes that hybridize poorly with (or with insufficient specificity for) target oligonucleotides. While several factors can affect a microarray's efficiency or success to facilitate genotyping—including temperature, salt concentration, probe or target size, probe or target concentration, among other factors—the nucleobase composition of an oligonucleotide probe significantly affects the probe's performance. In particular, an oligonucleotide probe's nucleobase composition affects whether the probe forms sufficiently strong hydrogen bonds with a target oligonucleotide.

When an oligonucleotide probe forms insufficient or weak hydrogen bonds with a target oligonucleotide, a washing solution rinses away the target oligonucleotide and potentially interferes with correctly determining a genomic sample's genotype. Because an oligonucleotide probe's nucleobase composition disables the probe from binding with the target nucleotide, an oligonucleotide probe with nucleobases that poorly compliment a target nucleotide can yield inaccurate genotyping results. Despite the presence of the target nucleotide in a genomic sample, therefore, a mismatched oligonucleotide probe can cause existing microarray systems to incorrectly determine that a target nucleotide (e.g., an SNP allele) is not present in a genomic sample.

To improve selection of probes for a microarray, some existing microarray systems use sophisticated software that can design and/or score oligonucleotide probes for particular target nucleotides. For instance, Illumina, Inc.® has developed a GenTrain® algorithm and GenTrain score that measures a calling quality of probes for SNPs detected by microarray. An existing microarray system can perform the GenTrain algorithm for oligonucleotide probes of different SNP alleles in part by measuring intensity values emitted by probes bound to target nucleotide fragments, clustering the intensity values according to different clustering models, selecting a clustering model, and determining GenTrain scores for the probes based on the relative intensity values and the selected clustering model. Such a GenTrain score generally measures an SNP calling quality of a probe, as described further by Shilin Zhao et al., “Strategies for Processing and Quality Control of Illumina Genotyping Arrays,” 19 Briefings in Bioinformatics 765-775 (2018), which is hereby incorporated in its entirety by reference. Despite having existing scores that indicate a probe's effectiveness in SNP calling or quality of clustering, existing microarray systems lack an effective way to directly account for the effect of a probe's nucleobase composition on either genotype calls or hybridization.

Because existing microarray systems often execute microarrays using inaccurate or indeterminate probes—and can only evaluate downstream effectiveness of a probe on genotyping—existing systems frequently re-run microarrays on multiple copies of DNA fragments from a genomic sample. Due to the same inaccuracies and downstream evaluation for probes, existing systems may also run different types of microarrays to determine more reliable genotyping calls for consensus. But such re-execution of microarrays or use of different microarray types can consume valuable computing resources on both specialized microarray devices and computing devices executing sequencing-data-analysis software—thereby performing redundant analyses and time-intensive-computer processing on such computing devices. Despite the importance of such microarrays, existing microarray systems cannot directly evaluate an impact of a probe's nucleotide sequence on genotype calls or hybridization before running (or re-running) an often laborious and computationally intensive microarray.

### SUMMARY

This disclosure describes one or more embodiments of systems, methods, and non-transitory computer readable storage media that solve one or more of the problems described above or provide other advantages over the art. In particular, the disclosed system uses a machine-learning model to classify or predict a probability of an oligonucleotide probe yielding an accurate genotype call or hybridizing with a target oligonucleotide—based on the oligonucleotide probe's nucleotide-sequence composition. To intelligently identify oligonucleotide probes that are more likely to yield accurate downstream genotyping—or more likely to successfully hybridize with target oligonucleotides—some embodiments of the disclosed machine-learning model include customized layers trained to detect motifs or other nucleotide-sequence patterns that correlate with favorable or unfavorable probe accuracy. By intelligently processing the nucleotide sequences of candidate oligonucleotide probes before implementing a microarray for a particular target oligonucleotide, the disclosed system can identify oligonucleotide probes with better genotyping accuracy (or better binding accuracy) than existing microarray systems for use in a microarray.

To illustrate but one embodiment, in some cases, the disclosed system identifies candidate oligonucleotide probes for hybridizing with target oligonucleotides and determines respective nucleotide sequences of one or more oligonucleotide probes from among the candidates. The disclosed system further uses a probe-classification-machine-learning model to determine probe accuracy classifications for particular oligonucleotide probes based on the particular oligonucleotide probes' nucleotide sequences. Such a probe accuracy classification may include a classification or score indicating a probability that the oligonucleotide probe (i) yields an accurate or an inaccurate genotype call or (ii) accurately or inaccurately binds to a target oligonucleotide for genotyping.

### BRIEF DESCRIPTION OF THE DRAWINGS

The detailed description refers to the drawings briefly described below.

FIG. 1 illustrates a computing-system environment in which a probe design system can operate in accordance with one or more embodiments of the present disclosure.

FIG. 2 illustrates a schematic diagram of the probe design system using a probe-classification-machine-learning model to determine probe accuracy classifications for oligonucleotide probes based on the oligonucleotide probe's nucleotide-sequence composition in accordance with one or more embodiments of the present disclosure.

FIGS. 3A-3B illustrate schematic diagrams of the probe design system categorizing candidate oligonucleotide probes into a favorable or unfavorable probe-accuracy-training class for training a probe-classification-machine-learning model based on threshold ranges for genotyping metrics in accordance with one or more embodiments of the present disclosure.

FIG. 4A illustrates the probe design system training a probe-classification-machine-learning model to determine probe accuracy classifications for oligonucleotide probes based on nucleotide sequences of the oligonucleotide probes in accordance with one or more embodiments of the present disclosure.

FIG. 4B illustrates the probe design system using a trained version of the probe-classification-machine-learning model to determine a probe accuracy classification for an oligonucleotide probe based on a nucleotide sequence of the oligonucleotide probe in accordance with one or more embodiments of the present disclosure.

FIG. 5 illustrates a graphical user interface for a probe design application on a computing device presenting recommended oligonucleotide probes for a microarray in accordance with one or more embodiments of the present disclosure.

FIGS. 6A-6B depict graphs showing true-positive and false-negative rates of a probe-classification-machine-learning model classifying oligonucleotide probes into probe accuracy classifications in accordance with one or more embodiments of the present disclosure.

FIG. 7 illustrate a series of acts for using a probe-classification-machine-learning model to determine probe accuracy classifications for oligonucleotide probes based on the oligonucleotide probe's nucleotide-sequence composition in accordance with one or more embodiments of the present disclosure.

FIG. 8 illustrate a series of acts for using a probe-classification-machine-learning model to determine different probe accuracy classifications for different oligonucleotide probes based on the different oligonucleotide probe's respective nucleotide-sequence compositions in accordance with one or more embodiments of the present disclosure.

FIG. 9 illustrates a block diagram of an example computing device in accordance with one or more embodiments of the present disclosure.

### DETAILED DESCRIPTION

This disclosure describes one or more embodiments of a probe design system that uses a machine-learning model to determine a probe accuracy classification for an oligonucleotide probe based on the oligonucleotide probe's nucleotide-sequence composition. Such a probe accuracy classification may include a score or other classification indicating a probability that the oligonucleotide probe (i) yields an accurate or an inaccurate genotype call for a target oligonucleotide or (ii) accurately or inaccurately binds to or hybridizes with the target oligonucleotide for genotyping. To intelligently score or otherwise classify oligonucleotide probes, in some cases, the probe design system uses a probe-classification-machine-learning model with customized layers trained to detect motifs or other nucleotide-sequence patterns that correlate with favorable or unfavorable probe accuracy based on genotyping metrics. The probe design system can accordingly train and implement a probe-classification-machine-learning model that facilitates more accurate oligonucleotide probes for a given microarray. As outlined below, the probe design system can effectively classify candidate oligonucleotide probes with accurate true-positive and false-negative rates—before consuming computing resources and specialized machines to implement a microarray for a particular target oligonucleotide.

As an overview, in some cases, the probe design system identifies candidate probes for hybridizing with target oligonucleotides. The probe design system further determines the nucleotide sequences of different candidate oligonucleotide probes to feed (as encoded data) to a probe-classification-machine-learning model. Based on encoded data representing the candidate oligonucleotide probes' nucleotide sequence, the probe-classification-machine-learning model determines a favorable probe accuracy classification for one subset of candidate oligonucleotide probes and, alternatively, an unfavorable probe accuracy classification for another subset of candidate oligonucleotide probes.

To train a probe-classification-machine-learning model, in some embodiments, the probe design system develops ground-truth classifications using genotyping metrics for candidate oligonucleotide probes. For instance, the probe design system identifies threshold ranges for genotyping metrics indicating accurate probes and inaccurate probes for genotyping and divides or categorizes training candidate oligonucleotide probes into favorable and unfavorable probe-accuracy-training classes based on the genotyping-metric threshold ranges. During training iterations, the probe-classification-machine-learning model predicts a probe accuracy classification (e.g., 0 or 1, score between 0 and 1) for a training oligonucleotide probe from those categorized into favorable and unfavorable probe-accuracy-training classes. Based on a comparison between the predicted probe accuracy classification and a ground-truth classification, the probe design system adjusts parameters for the probe-classification-machine-learning model.

As suggested above, in some implementations, the probe-classification-machine-learning model includes layers designed and trained to detect motifs or other nucleotide-sequence patterns that correlate with favorable or unfavorable probe accuracy. For instance, in some cases, the probe-classification-machine-learning model includes filters of a kernel size customized for nucleotide-sequence-pattern recognition. Additionally, or alternatively, the probe-classification-machine-learning model includes channels customized for nucleobase-class recognition (e.g., A, T, C, G).

In addition to training, the probe design system can implement a trained version of the probe-classification-machine-learning model to determine probe accuracy classifications for input oligonucleotide probes. For instance, the probe-classification-machine-learning model can determine a score indicating a genotyping probability that a given oligonucleotide probe yields an accurate genotype call or a binding probability that the given oligonucleotide probe accurately binds to a target oligonucleotide for genotyping. In addition or in the alternative to a score, the probe-classification-machine-learning model can determine binary or ternary probe accuracy classifications, including a favorable probe accuracy class and an unfavorable probe accuracy class. Such binary probe accuracy classifications may include (i) a favorable or unfavorable genotyping accuracy class indicating a probability that the oligonucleotide probe yields an accurate or inaccurate genotype call or (ii) a favorable or unfavorable binding accuracy class indicating a probability that the oligonucleotide probe accurately or inaccurately binds to a target oligonucleotide for genotyping.

Based on probe accuracy classifications for oligonucleotide probes, in some embodiments, the probe design system generates probe recommendations for microarrays. Based on probe accuracy classifications, for instance, the probe design system can select one or more oligonucleotide probes (or recommend against one or more oligonucleotide probes) for use in a microarray. After selection, the probe design system can likewise facilitate performing a microarray using recommended oligonucleotide probes.

As indicated above, the probe design system provides several technical advantages relative to existing sequencing systems, such as by improving the accuracy of probe hybridization or genotyping calls in microarrays and improving microarray computing efficiency. For instance, in some embodiments, the probe design system improves the accuracy with which selected oligonucleotide probes hybridize with target nucleotides as part of a genotyping microarray. As suggested above, existing microarray systems frequently use oligonucleotide probes that form weak or insufficient bonds with corresponding target nucleotides and, consequently, wash away during a microarray, thereby compromising the accuracy of a microarray. As discovered by the inventors of this disclosure, however, when a trained machine-learning model scores or otherwise classifies a probe accuracy of a candidate oligonucleotide probe—based on a nucleotide sequence of the candidate oligonucleotide probe—the probe design system can identify oligonucleotide probes exhibiting superior hybridization accuracy than probes designed or selected by existing microarray systems. Indeed, in some embodiments, the disclosed probe-classification-machine-learning model determines a favorable or unfavorable binding accuracy classification for an oligonucleotide probe indicating a probability that the oligonucleotide probe accurately or inaccurately binds to a target oligonucleotide for genotyping. In a first-of-its-kind machine-learning model, the disclosed probe-classification-machine-learning model can use layers trained to identify nucleotide-sequence patterns to identify more accurate probes before performing a microarray.

As further noted above, existing microarray systems use oligonucleotide probes with nucleobase compositions that disable certain probes from binding target nucleotide and that yield inaccurate genotype calls. In contrast to such existing microarray systems, by scoring or otherwise classifying a probe accuracy of a candidate oligonucleotide probe based on a nucleotide sequence of the candidate oligonucleotide probe, the probe design system can identify oligonucleotide probes exhibiting superior genotyping accuracy than existing microarray systems. Indeed, in some embodiments, the disclosed probe-classification-machine-learning model determines a favorable or unfavorable genotyping accuracy class for an oligonucleotide probe indicating a probability that the oligonucleotide probe yields an accurate or inaccurate genotype call.

In part due to such improved probe accuracy, in certain implementations, the probe design system improves the computing efficiency and processing time consumed by specialized sequencing devices and/or computing devices running microarrays. As noted above, some existing systems re-run microarrays on multiple copies of DNA fragments from a genomic sample or run different types of microarrays to determine more reliable genotyping calls. Rather than perform redundant or time-intensive processing on specialized sequencing devices, the probe design system can apply a probe-classification-machine-learning model to nucleotide sequences of candidate oligonucleotide probes for a microarray and identify oligonucleotide probes with nucleotide sequences compatible with accurate genotyping and/or accurate hybridization—thereby obviating microarray re-runs or diversified microarray types. By introducing the disclosed first-of-its-kind machine-learning model, in some embodiments, the probe design system efficiently identifies accurate oligonucleotide probes for specific target nucleotides and avoids a drawn-out back-and-forth of using multiple microarrays on microarray devices.

As illustrated by the foregoing discussion, the present disclosure utilizes a variety of terms to describe features and advantages of the probe design system. As used herein, for example, the term “oligonucleotide probe” refers to a fragment of DNA designed to complement and hybridize with a nucleotide sequence of a target oligonucleotide. An oligonucleotide probe generally hybridizes with a target oligonucleotide by forming hydrogen bonds. In some cases, an oligonucleotide probe includes a single-stranded fragment of DNA of approximately 15 to 1,000 nucleobases in length to which a label and/or a slide or chip can be attached for a microarray. Accordingly, an oligonucleotide probe can comprise (or be attached to) a chemical tag, fluorescent tag, radioactive tag, or other label that emits a signal captured or otherwise detected by a camera, imaging device, or other scanner. For instance, when an enzyme incorporates labelled nucleobases into an oligonucleotide probe and extends the oligonucleotide probe to compliment the target oligonucleotide, the oligonucleotide probe incorporates a tag or label that can emit light or other signal.

Relatedly, the term “microarray” refers to an assay using oligonucleotide probes attached to a slide or chip to detect a presence of target nucleotides corresponding to one or more genomic samples. For instance, a microarray includes an assay comprising a collection of oligonucleotide probes, attached to spots or beads on a surface of a slide or chip, that detect a presence or absence of target oligonucleotides by binding to or hybridizing with such target oligonucleotides. By detecting signals from labels attached to oligonucleotide probes bound to target nucleotides—and sometimes comparing signals from labels under control conditions—a microarray can detect a presence or absence of one or more target nucleotides from one or more genomic samples. Such target oligonucleotides may represent some or all of a gene, promoter region, or other nucleotide sequence from a genomic sample.

As just indicated, an oligonucleotide probe is designed to complement target oligonucleotides. As used herein, the term “target oligonucleotide” refers to a nucleotide sequence selected from one or more genomic samples for detection by assay. In some cases, a target oligonucleotide constitutes a sequence of nucleotides isolated or extracted from a sample organism (or a copy of such an isolated or extracted sequence) for detection by a microarray. In particular, a target oligonucleotide includes a segment of a nucleic acid polymer, such as a DNA fragment, that is isolated or extracted from a genomic sample, composed of nitrogenous heterocyclic bases. In some cases, the nucleic acid polymer is transformed into an oligonucleotide of complimentary DNA (cDNA). For instance, a target oligonucleotide includes a nucleotide sequence representing some or all of a gene, a promoter region, a motif, or other selected nucleotide sequence subject to an assay.

As further used herein, the term “genotype call” refers to a determination or prediction of a particular genotype of a genomic sample at a genomic site. In particular, a genotype call can include a prediction of a particular genotype of a genomic sample with respect to a reference sample or reference genome at a genomic coordinate or a genomic region. A genotype call is often determined for a genomic coordinate or genomic region at which an SNP or other variant has been identified for a population of organisms.

Relatedly, the term “genotyping metric” refers to a quantitative measurement or score indicating a quality, regularity, or error-rate of a genotype call or light signal associated with an oligonucleotide probe. For instance, a genotyping metric includes a quantitative measurement or score indicating a degree to which (i) a genotype call associated with an oligonucleotide probe is accurate or reflects accurately separated clusters of intensity values, (ii) genotype calls are determined or not determined associated with the oligonucleotide probe, (iii) intensity values for light signals emitted from labels attached to the oligonucleotide probe (e.g., incorporated labeled nucleobases complimenting nucleobases of a target nucleotide) are separated by clusters or conform to a norm, (iv) genotype calls reflect an error that is inconsistence with allelic inheritance patterns, or (v) genotype calls associated with the oligonucleotide probe can be reproduced.

As used herein, the term “machine-learning model” refers to a computer algorithm or a collection of computer algorithms that automatically improve performing a particular task through experience based on use of data. For example, a machine-learning model can utilize one or more learning techniques to improve in accuracy and/or effectiveness. Example machine-learning models include various types of decision trees, support vector machines, Bayesian networks, or neural networks. In some cases, a probe-classification-machine-learning model constitutes a deep neural network (e.g., convolutional neural network) or a series of decision trees (e.g., random forest, XGBoost), while in other cases the probe-classification-machine-learning model constitutes a multilayer perceptron, a linear regression, a support vector machine, a deep tabular learning architecture, a deep learning transformer (e.g., self-attention-based-tabular transformer), or a logistic regression.

In some cases, the probe design system utilizes a probe-classification-machine-learning model to classify or predict accuracy probabilities for an oligonucleotide probe. As used herein, the term “probe-classification-machine-learning model” refers to a machine-learning model that determines a value indicating whether an oligonucleotide probe will yield an accurate genotype call or hybridize with a target oligonucleotide. For example, in some cases, the probe-classification-machine-learning model is trained to generate probe accuracy classifications for particular oligonucleotide probes based on the particular oligonucleotide probes' nucleotide sequence. A probe-classification-machine-learning model can take the form of a neural network, a collection of decision trees, or other structures noted above. In certain implementations, a probe-classification-machine-learning model includes customized layers trained to detect motifs or other nucleotide-sequence patterns that correlate with favorable or unfavorable probe accuracy based on genotyping metrics.

Relatedly, the term “probe accuracy classification” refers to a label, score, or metric indicating an accuracy of an oligonucleotide probe for genotyping. In particular, a probe accuracy classification includes a label, score, or metric indicating a probability or likelihood that an oligonucleotide probe (i) yields an accurate or an inaccurate genotype call for a target oligonucleotide or (ii) accurately or inaccurately binds to or hybridizes with the target oligonucleotide for genotyping. Accordingly, a probe accuracy classification for an oligonucleotide probe can include a favorable genotyping accuracy class indicating a probability that the oligonucleotide probe yields an accurate genotype call or an unfavorable genotyping accuracy class indicating a probability that the oligonucleotide probe yields an inaccurate genotype call. Further, a probe accuracy classification for an oligonucleotide probe can include a favorable binding accuracy class indicating a probability that the oligonucleotide probe accurately binds to a target oligonucleotide for genotyping or an unfavorable binding accuracy class indicating a probability that the oligonucleotide probe inaccurately binds to the target oligonucleotide for genotyping. As indicated above, a probe accuracy classification can also be a score (e.g., a value between 0 and 1 indicating probability); a ternary classification of high probe accuracy, medium probe accuracy, or low probe accuracy; a quaternary classification of high probe accuracy, medium probe accuracy, indeterminate probe accuracy, or low probe accuracy; or another multi-part classification (e.g., quinary classification, senary classification).

Relatedly, the term “nucleobase class” refers to a particular type or kind of nitrogenous base. For instance, a genome or nucleotide sequence may include five different nucleobase classes, including adenine (A), cytosine (C), guanine (G), or thymine (T), or uracil (U).

As further used herein, the term “genomic coordinate” (or sometimes simply “coordinate”) refers to a particular location or position of a nucleobase within a genome (e.g., an organism's genome or a reference genome). In some cases, a genomic coordinate includes an identifier for a particular chromosome of a genome and an identifier for a position of a nucleobase within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleobase within the source for the reference genome (e.g., mt:16568 or SARS-CoV-2:29001). By contrast, in certain cases, a genomic coordinate refers to a position of a nucleobase within a reference genome without reference to a chromosome or source (e.g., 29727).

As mentioned above, a “genomic region” refers to a range of genomic coordinates. Like genomic coordinates, in certain embodiments, a genomic region may be identified by an identifier for a chromosome and a particular position or positions, such as numbered positions following the identifier for a chromosome (e.g., chr1:1234570-1234870).

As further used herein, a “pattern” or “pattern within a nucleotide sequence” refers to a repeated or distinctive sequence of nucleobases. For instance, a pattern within a nucleotide sequence can include homopolymers of a same nucleotide base, a guanine quadruplex, a dinucleotide-repeat sequence, a tri-nucleotide-repeat sequence, an inverted-repeat sequence, a minisatellite sequence, a microsatellite sequence, a palindromic sequence, or other sequence.

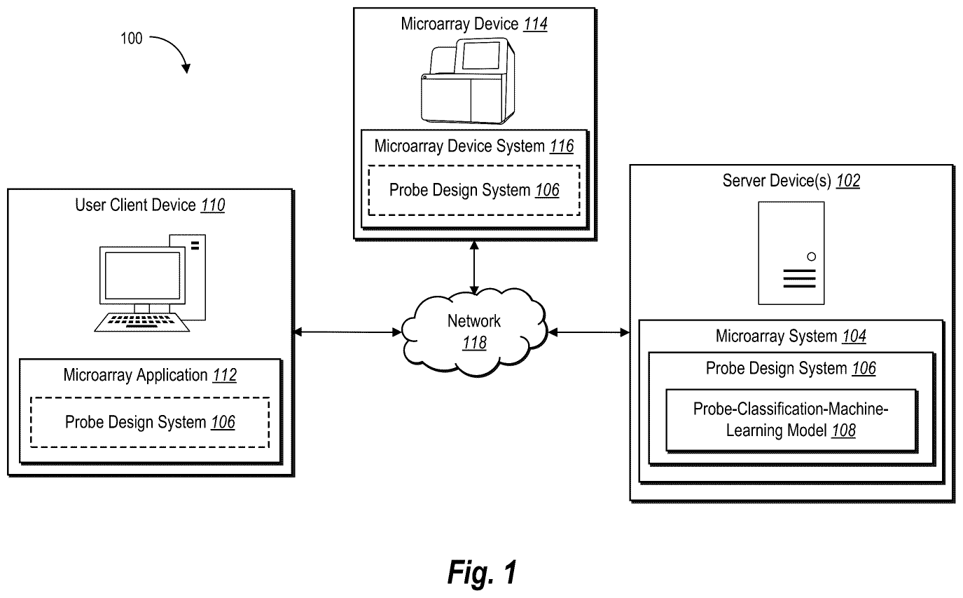

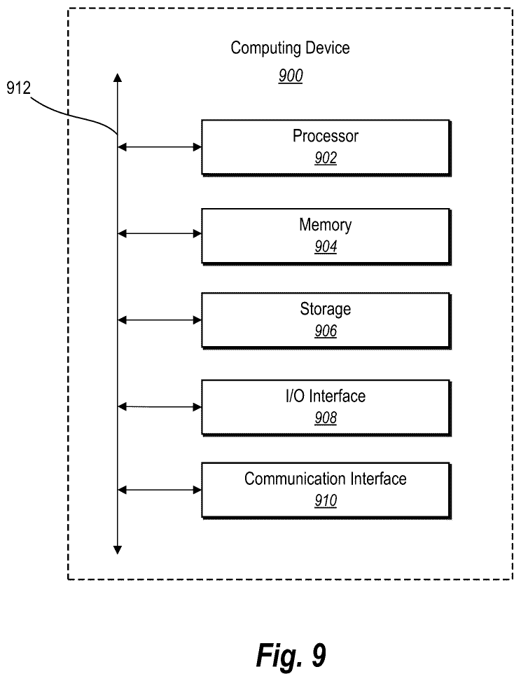

The following paragraphs describe the probe design system with respect to illustrative figures that portray example embodiments and implementations. For example, FIG. 1 illustrates a schematic diagram of a computing system 100 in which a probe design system 106 operates in accordance with one or more embodiments. As illustrated, the computing system includes server device(s) 102, a microarray device 114, and a user client device 110 connected via a network 118. While FIG. 1 shows an embodiment of the probe design system 106, this disclosure describes alternative embodiments and configurations below. As shown in FIG. 1, the microarray device 114, the server device(s) 102, and the user client device 110 can communicate with each other via the network 118. The network 118 comprises any suitable network over which computing devices can communicate. Example networks are discussed in additional detail below with respect to FIG. 9.

As shown in FIG. 1, the server device(s) 102 comprise the probe design system 106 and a probe-classification-machine-learning model 108. When executed by the server device(s) 102 or another computing device shown in FIG. 1, the probe design system 106 identifies candidate oligonucleotide probes for hybridizing with target oligonucleotides and determines respective nucleotide sequences of one or more oligonucleotide probes from among the candidates. The probe design system 106 further uses the probe-classification-machine-learning model 108 to determine probe accuracy classifications for particular oligonucleotide probes based on the particular oligonucleotide probes' nucleotide sequences. Based on the probe accuracy classifications, in some embodiments, the probe design system 106 selects and recommends oligonucleotide probes for use in a microarray from among candidate oligonucleotide probes. By sending data representing the probe accuracy classifications and/or recommended oligonucleotide probes to the user client device 110, the user client device 110 can present a graphical user interface comprising one or both of probe accuracy classifications for candidate oligonucleotide probes and recommended oligonucleotide probes.

As indicated by FIG. 1, the microarray device 114 comprises a microarray device system 116 for performing a microarray to detect a presence or absence of target nucleotides from a genomic sample. By performing a microarray, the microarray device 114 may analyze extracted sample nucleotide sequences from a genomic sample to detect particular genes, variants, or other target nucleotides from the genomic sample. In some embodiments, by executing the microarray device system 116, the microarray device 114 introduces, to a slide or a chip comprising oligonucleotide probes with labels, a solution comprising extracted nucleotide sequences from a genomic sample and a control sample. For instance, the microarray device 114 receives slides (e.g., lab-on-a chip) with oligonucleotide probes that are attached to various spots of the slides and designed to hybridize with corresponding target nucleotides.

After introducing extracted sample nucleotide sequences to a slide under target conditions (e.g., specific temperature and salt concentration), the oligonucleotide probes hybridize with target nucleotide probes. During the microarray, the microarray device 114 can incorporate a fluorescently labeled nucleobase that extends the oligonucleotide probe and that compliments a nucleobase of the target oligonucleotide as a template. Accordingly the microarray device 114 can incorporate labeled nucleobase by labeled nucleobase to extend the oligonucleotide probe and compliment the target oligonucleotide during the microarray. In some cases, the microarray device 114 uses labeled antibodies that include a fluorescent label to enhance the fluorescent light or signal emitted by an oligonucleotide probe. After washing the slide or chip to discard unhybridized nucleotides and reagents, the microarray device 114 scans the surface of the slide or chip with a camera to detect whether the nucleotide sequences extracted from the genomic sample (and control sample) hybridize with the labeled oligonucleotide probes and, consequently, extend the oligonucleotide probe with labelled nucleobases complimenting the target oligonucleotide.

In a two-color microarray, a camera of the microarray device 114 captures light signals emitted from a first label of one color attached to a first oligonucleotide probe hybridized with a target and a second label of another color attached to a second oligonucleotide probe hybridized with a control sample. In some cases, the different colors correspond to oligonucleotide probes that hybridize with different alleles. In a single-color or single-label microarray, the camera of the microarray device 114 captures light signals emitted from labels of a same color from oligonucleotide probes hybridized with target nucleotide sequences of the genomic sample and oligonucleotide probes hybridized with nucleotide sequences of the control sample.

When the scan detects a light emitted by the labeled oligonucleotide probes, in some cases, the microarray device system 116 sends metrics indicating intensity values of the emitted light and corresponding locations to a microarray system 104. Based on the intensity values and locations corresponding to oligonucleotide probes or controls, the microarray system 104 (i) determines whether target oligonucleotides corresponding to the oligonucleotide probes are present or absent in the sample nucleotide sequences extracted from the genomic sample and (ii) generate corresponding genotype calls for the target oligonucleotides.

In some cases, the server device(s) 102 is located at or near a same physical location of the microarray device 114 or remotely from the microarray device 114. Indeed, in some embodiments, the server device(s) 102 and the microarray device 114 are integrated into a same computing device. The server device(s) 102 may run a microarray system 104 or the probe design system 106 to generate, receive, analyze, store, and transmit digital data, such as by receiving intensity-value data or determining variant calls based on analyzing such intensity-value data.

As suggested by FIG. 1, the microarray device 114 may send (and the server device(s) 102 may receive) intensity-value data generated during a microarray of the microarray device 114. By executing software in the form of the microarray system 104 or the probe design system 106, the server device(s) 102 may determine genotype calls for target oligonucleotides of a genomic sample at particular genomic coordinates. The server device(s) 102 may also communicate with the user client device 110. In particular, the server device(s) 102 can send data to the user client device 110, including a variant call file (VCF), or other information indicating nucleobase calls or other metrics.

In some embodiments, the server device(s) 102 comprise a distributed collection of servers where the server device(s) 102 include a number of server devices distributed across the network 118 and located in the same or different physical locations. Further, the server device(s) 102 can comprise a content server, an application server, a communication server, a web-hosting server, or another type of server.

As further illustrated and indicated in FIG. 1, the user client device 110 can generate, store, receive, and send digital data. In particular, the user client device 110 can receive variant calls from the server device(s) 102 or receive intensity-value data from the microarray device 114. Furthermore, the user client device 110 may communicate with the server device(s) 102 or the server device(s) 102 to receive a VCF comprising nucleobase calls and/or other metrics, such as a base-call-quality metrics or pass-filter metrics. The user client device 110 can accordingly present or display information pertaining to variant calls or other nucleobase calls within a graphical user interface to a user associated with the user client device 110. In particular, the user client device 110 can present results from a microarray or recommended oligonucleotide probes for a particular microarray.

Although FIG. 1 depicts the user client device 110 as a desktop or laptop computer, the user client device 110 may comprise various types of client devices. For example, in some embodiments, the user client device 110 includes non-mobile devices, such as desktop computers or servers, or other types of client devices. In yet other embodiments, the user client device 110 includes mobile devices, such as laptops, tablets, mobile telephones, or smartphones. Additional details regarding the user client device 110 are discussed below with respect to FIG. 9.

As further illustrated in FIG. 1, the user client device 110 includes a microarray application 112. The microarray application 112 may be a web application or a native application stored and executed on the user client device 110 (e.g., a mobile application, desktop application). The microarray application 112 can include instructions that (when executed) cause the user client device 110 to receive data from the probe design system 106 and present, for display at the user client device 110, intensity-value data from a microarray, probe accuracy classifications from the probe-classification-machine-learning model 108, or recommend oligonucleotide probes for a microarray.

As further illustrated in FIG. 1, a version of the probe design system 106 may be located on the user client device 110 as part of the microarray application 112 or on the microarray device 114. Accordingly, in some embodiments, the probe design system 106 is implemented by (e.g., located entirely or in part) on the user client device 110. In yet other embodiments, the probe design system 106 is implemented by one or more other components of the computing system 100, such as the microarray device 114. In particular, the probe design system 106 can be implemented in a variety of different ways across the microarray device 114, the user client device 110, and the server device(s) 102. For example, the probe design system 106 can be downloaded from the server device(s) 102 to the microarray device 114 and/or the user client device 110 where all or part of the functionality of the probe design system 106 is performed at each respective device within the computing system.

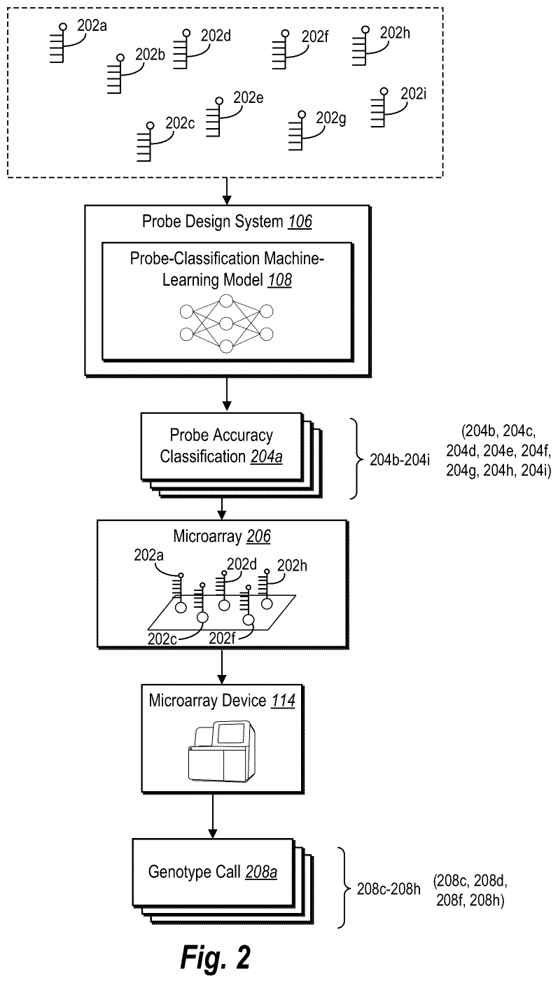

As indicated above, the probe design system 106 can use a probe-classification-machine-learning model to determine probe accuracy classifications. In accordance with one or more embodiments, FIG. 2 illustrates an example of the probe design system 106 (i) identifying candidate oligonucleotide probes, (ii) using a probe-classification-machine-learning model to determine probe accuracy classifications for the candidate oligonucleotide probes, and (iii) optionally performing a microarray using recommended oligonucleotide probes based on the probe accuracy classifications.

As shown in FIG. 2, for instance, the probe design system 106 identifies candidate oligonucleotide probes 202a, 202b, 202c, 202d, 202e, 202f, 202g, 202h, and 202i. In some embodiments, the probe design system 106 receives data indications from the user client device 110 selecting one or more of the candidate oligonucleotide probes 202a-202i. Additionally, or alternatively, the probe design system 106 receives a dataset representing one or more of the candidate oligonucleotide probes 202a-202i from an existing set of probes, such as a set of probes from a commercially available microarray kit.

Regardless of how the candidate oligonucleotide probes 202a-202i are identified, in some embodiments, the probe design system 106 receives a dataset representing individual nucleotide sequences (nucleobase by nucleobase) of the candidate oligonucleotide probes 202a-202i. Such a dataset may come from an existing data file comprising data representations of the individual nucleotide sequences (e.g., entries of A, T, C, G) or from data entry by the user client device 110 representing the individual nucleotide sequences. In some embodiments, the probe design system 106 determines the individual nucleotide sequences of the candidate oligonucleotide probes 202a-202i from such received datasets.

As indicated above, in some cases, the candidate oligonucleotide probes 202a-202i correspond to target oligonucleotides that represent to some or all of a gene, promoter region, or other nucleotide sequence. Accordingly, in addition to an individual nucleotide sequence, in some embodiments, the probe design system 106 receives data representing a genomic region or genomic coordinates for target nucleotides corresponding to the candidate oligonucleotide probes 202a-202i.

After identifying the candidate oligonucleotide probes 202a-202i and determining the nucleotide sequences of the candidate oligonucleotide probes 202a-202i, in certain implementations, the probe design system 106 sequentially inputs datasets representing the nucleotide sequences of the candidate oligonucleotide probes 202a-202i into the probe-classification-machine-learning model 108. To process the candidate oligonucleotide probes 202a-202i, the probe design system 106 converts (or uses an existing dataset representing) a candidate oligonucleotide probe from the candidate oligonucleotide probes 202a-202i into a matrix, feature vector, or feature map representing the nucleotide-sequence composition.

As noted above, in some embodiments, the probe-classification-machine-learning model 108 includes layers designed and trained to (i) detect different nucleobase classes from the input matrix, vector, or feature map or (ii) detect motifs or other nucleotide-sequence patterns that correlate with favorable or unfavorable probe accuracy. For instance, in some cases, the probe-classification-machine-learning model 108 comes in a form of a neural network that includes a number of channels customized for nucleobase-class recognition (e.g., A, T, C, G) and adjusted to avoid overfitting training data. Additionally, or alternatively, the probe-classification-machine-learning model 108 comprises filters of a kernel size customized for nucleotide-sequence-pattern recognition, such as a dinucleotide-repeat sequence, a tri-nucleotide-repeat sequence, or, alternatively, a motif or other pattern detected across multiple windows of multiple nucleobases (e.g., kernel size for analyzing 3, 4, or more nucleobases) from an oligonucleotide probe's nucleotide sequence.

Based on the datasets representing the candidate oligonucleotide probes 202a-202i, as further shown in FIG. 2, the probe-classification-machine-learning model 108 generates probe accuracy classifications 204a-204i, respectively. Based on a dataset representing the candidate oligonucleotide probe 202a, for instance, the probe-classification-machine-learning model 108 generates the probe accuracy classification 204a. As indicated above, in some embodiments, the probe accuracy classification 204a indicates a probability or likelihood that the candidate oligonucleotide probe 202a (i) yields an accurate or an inaccurate genotype call for a target oligonucleotide or (ii) accurately or inaccurately binds to or hybridizes with the target oligonucleotide for genotyping.

Because the probe accuracy classifications 204a-204i quantify accuracy probabilities for different candidate oligonucleotide probes, the probe accuracy classifications 204a-204i may individually represent different probabilities or classes for their respective candidate oligonucleotide probes 202a-202i. Accordingly, in some cases, the probe accuracy classifications 204a-204i comprise (i) values ranging from 0 to 1 or (ii) comprise favorable probe accuracy classifications and unfavorable probe accuracy classifications. As indicated above, the probe accuracy classifications 204a-204i can take various other forms (e.g., ternary classifications).

Based on the probe accuracy classifications 204a-204i, in some embodiments, the probe design system 106 selects (or receives selections of) oligonucleotide probes from the candidate oligonucleotide probes 202a-202i for use in a microarray 206. As indicated by FIG. 2, for instance, the probe design system 106 recommends (or receives a selection from the user client device 110 of) the candidate oligonucleotide probes 202a, 202c, 202d, 202f, and 202h—based on the probe accuracy classifications 204a, 204c, 204d, 204f, and 204h indicating better accuracy than the probe accuracy classifications 204b, 204e, 204g, and 204i.

For instance, if the probe accuracy classifications 204a, 204c, 204d, 204f, and 204h constitute favorable accuracy classifications—and the probe accuracy classifications 204b, 204e, and 204g constitute unfavorable accuracy classifications—the probe design system 106 selects the candidate oligonucleotide probes 202a, 202c, 202d, 202f, and 202h with favorable accuracy. As a further example, if the probe accuracy classifications 204a, 204c, 204d, 204f, and 204h constitute values above a probability threshold—and the probe accuracy classifications 204b, 204e, and 204g constitute values below a probability threshold—the probe design system 106 selects the candidate oligonucleotide probes 202a, 202c, 202d, 202f, and 202h as more likely to yield accurate genotype calls for a target oligonucleotide or to accurately bind to the target oligonucleotide for genotyping.

As further shown in FIG. 2, in certain implementations, the microarray device 114 together with the microarray device system 116 perform the microarray 206 using the candidate oligonucleotide probes 202a, 202c, 202d, 202f, and 202h. In some embodiments, the microarray system 104 receives a slide or chip comprising one or more copies of the candidate oligonucleotide probes 202a, 202c, 202d, 202f, and 202h attached to wells or beads. When performed on the microarray device 114, specific target oligonucleotides from one or more genomic samples hybridize with the one or more copies of the candidate oligonucleotide probes 202a, 202c, 202d, 202f, and 202h. Such target oligonucleotides may correspond to a gene with a candidate SNP or other candidate variant, part of a gene with a candidate SNP or other candidate variant, or other nucleotide sequence located at one or more genomic coordinates of a reference genome.

Based on light detected (or not detected) from labels of the candidate oligonucleotide probes 202a, 202c, 202d, 202f, and 202h, the microarray system 104 determines genotype calls 208a, 208c, 208d, 208f, and 208h, respectively. In some cases, the genotype calls 208a-208h correspond to specific genomic regions or coordinates (as indicated above) and represent SNPs, indels, or other variants. Because the probe design system 106 determines the probe accuracy classifications 204a-204i and identifies more accurate oligonucleotide probes before the microarray 206, the probe design system 106 intelligently and efficiently identifies oligonucleotide probes that are more likely to yield accurate genotyping calls and less likely to require re-running a microarray or using another microarray to support genotype calls.

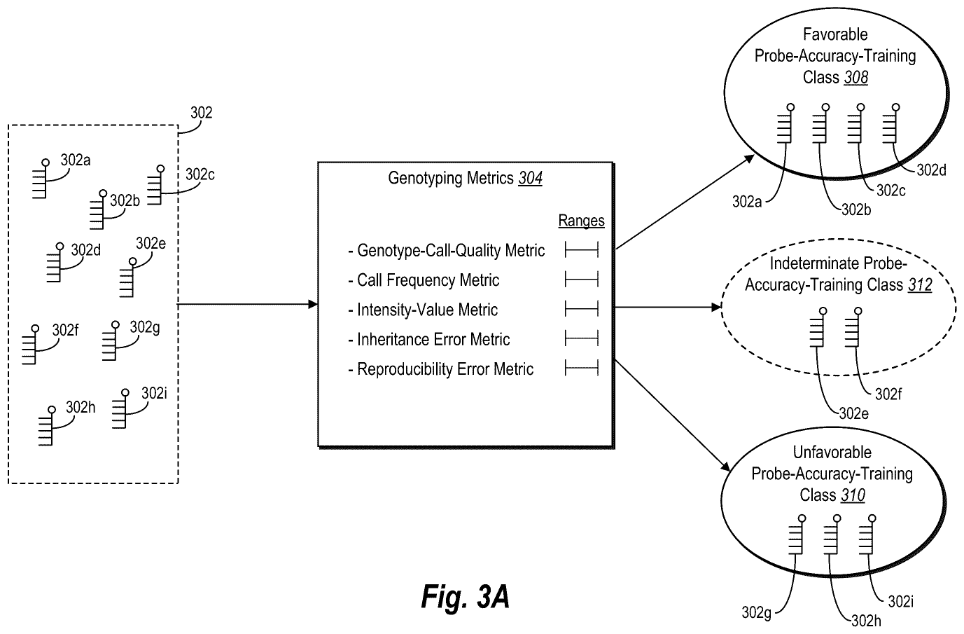

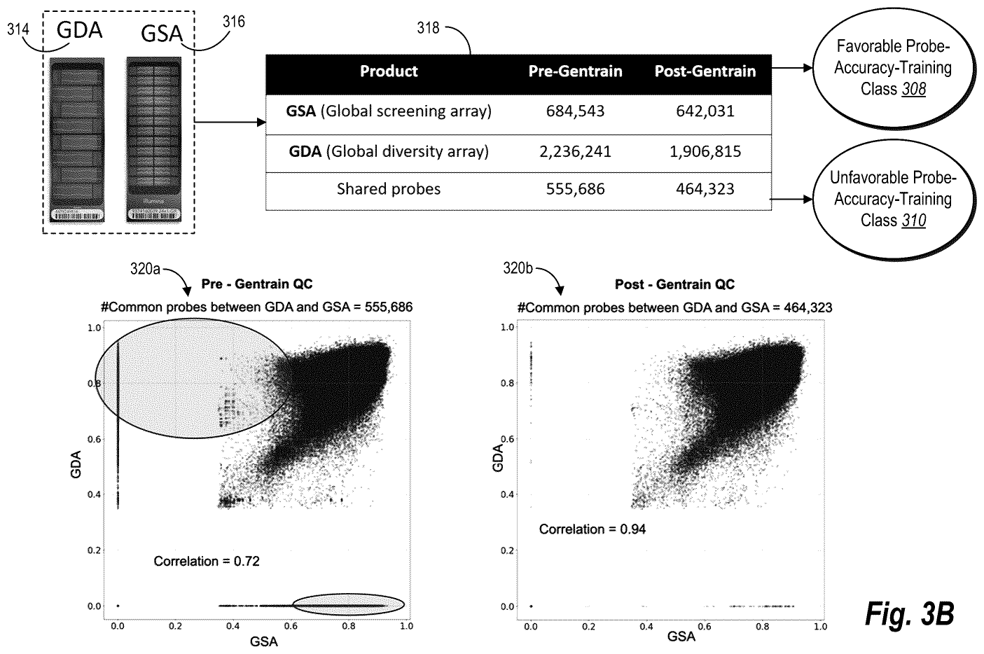

As indicated above, in some cases, the probe design system 106 develops ground-truth classifications using threshold ranges of genotyping metrics for candidate oligonucleotide probes. In accordance with one or more embodiments, FIG. 3A illustrates the probe design system 106 identifying threshold ranges for genotyping metrics associated with candidate oligonucleotide probes and categorizing training candidate oligonucleotide probes into favorable and unfavorable probe-accuracy-training classes based on the genotyping-metric threshold ranges. In accordance with one or more embodiments, FIG. 3B illustrates the probe design system 106 categorizing training candidate oligonucleotide probes for existing microarrays into favorable and unfavorable probe-accuracy-training classes based on the threshold ranges for genotyping metrics.

As shown in FIG. 3A, for instance, the probe design system 106 identifies a set of candidate oligonucleotide probes 302 comprising candidate oligonucleotide probes 302a-302i for training a probe-classification-machine-learning model. In some cases, the probe design system 106 receives a dataset representing the candidate oligonucleotide probes 302a-302i from an existing microarray. As described below, for instance, the probe design system 106 can receive a dataset representing candidate oligonucleotide probes for one or both of a Global Screen Array (GSA) and a Global Diversity Array (GDA) microarrays available from Illumina, Inc. Additionally, or alternatively, in some embodiments, the probe design system 106 receives data from the user client device 110 indicating selections of one or more of the candidate oligonucleotide probes 302a-302i. For purposes of illustration and space constraints, FIG. 3A depicts the candidate oligonucleotide probes 302a-302i as merely an example number of candidate oligonucleotide probes. As further indicated below, the probe design system 106 may analyze hundreds, thousands, or more candidate oligonucleotide probes for training or implementing a probe-classification-machine-learning model.

In addition to identifying the candidate oligonucleotide probes 302a-302i, in certain implementations, the probe design system 106 determines or identifies threshold ranges for genotyping metrics associated with the candidate oligonucleotide probes 302a-302i. As indicated above, a genotyping metric represents a quantitative measurement or score indicating a quality, regularity, or error rate of a genotype call or light signal associated with a candidate oligonucleotide probe. To categorize the candidate oligonucleotide probes 302a-302i into a favorable probe-accuracy-training class 308 and an unfavorable probe-accuracy-training class 310, in certain cases, the probe design system 106 uses threshold ranges for one or more of genotyping metrics 304. As depicted in FIG. 3A, the genotyping metrics 304 may include one or more of genotype-call-quality metrics, call frequency metrics, intensity-value metrics, inheritance error metrics, or reproducibility error metrics. To clearly categorize the candidate oligonucleotide probes 302a-302i, for instance, the probe design system 106 can identify upper limits of one or more genotyping metrics for categorizing a candidate oligonucleotide probe into the favorable probe-accuracy-training class 308 and lower limits of one or more genotyping metrics for categorizing a candidate oligonucleotide probe into the unfavorable probe-accuracy-training class 310.

As set forth in more detail below, in some cases, genotype-call-quality metrics include one or more of GenTrain scores, a quantile of GenCall scores, or NextGen1 scores; a call frequency metric includes scores or metrics indicating a percentage of samples at a particular locus (e.g., genomic coordinate) for which an oligonucleotide probe resulted a genotype call; intensity-value metrics include one or more of average R intensity values or cluster separation scores; inheritance error metrics include one or more of parent-child (PC) errors or parent-parent-child (PPC) errors; and reproducibility error metrics includes values indicating a reproducibility of genotype calls for replicate genomic samples at each variant genomic coordinate.

As indicated above, the probe design system 106 can identify threshold ranges for genotype-call-quality metrics to use for categorizing candidate oligonucleotide probes. For instance, the probe design system 106 identifies threshold ranges for GenTrain score that measure a genotyping calling quality of oligonucleotide probes for SNPs detected by microarray—based on clustering of intensity values emitted by oligonucleotide probes bound to target oligonucleotides. In particular, in some cases, a GenTrain score can range from 0 to 1 and measure a quality with which an SNP intensity-value graph conforms to standard or expected positions of three intensity-value clusters. In some cases, the probe design system 106 identifies (i) an upper limit of 0.85 for a GenTrain score above or equal to which a candidate oligonucleotide probe satisfies one threshold for the favorable probe-accuracy-training class 308 and (ii) a lower limit of 0.3 for a GenTrain score below or equal to which a candidate oligonucleotide probe satisfies one threshold for the unfavorable probe-accuracy-training class 310. As indicated below, in some embodiments, the probe design system 106 uses multiple threshold ranges from multiple genotyping metrics for categorizing candidate oligonucleotide probes into the favorable probe-accuracy-training class 308 or the unfavorable probe-accuracy-training class 310.

As a further example of a threshold range for a genotype-call-quality metric, in some embodiments, the probe design system 106 identifies threshold ranges for a particular quantile of GenCall score associated with a candidate oligonucleotide probe. For instance, a GenCall score quantifies a quality of a genotype call ranging from 0 to 1 associated with a candidate oligonucleotide probe. Relatedly, a 10% GenCall score represents the 10% quantile of GenCall scores associated with a candidate oligonucleotide probe. In some cases, the probe design system 106 identifies (i) an upper limit of 0.6 for 10% GenCall scores above or equal to which a candidate oligonucleotide probe satisfies one threshold for the favorable probe-accuracy-training class 308 and (ii) a lower limit of 0.3 for 10% GenCall scores below or equal to which a candidate oligonucleotide probe satisfies one threshold for the unfavorable probe-accuracy-training class 310.

As yet a further example of a threshold range for a genotype-call-quality metric, in some embodiments, the probe design system 106 identifies threshold ranges for a NextGen1 score associated with a candidate oligonucleotide probe. A NextGen1 score indicates a holistic quality of a performance for an oligonucleotide probe's performance in yielding an SNP call. As long as an SNP is not monomorphic or condensed, a NextGen1 score can be useful for evaluating the performance of oligonucleotide probes for SNPs. In some cases, a NextGen1 score combines multiple genotype-call-quality metrics, call frequency metrics, intensity-value metrics, inheritance error metrics, and reproducibility error metrics. In some cases, the probe design system 106 identifies (i) an upper limit of 0.7 for NextGen1 scores above or equal to which a candidate oligonucleotide probe satisfies one threshold for the favorable probe-accuracy-training class 308 and (ii) a lower limit of 0.7 for NextGen1 scores below or equal to which a candidate oligonucleotide probe satisfies one threshold for the unfavorable probe-accuracy-training class 310.

As further indicated above, the probe design system 106 can identify threshold ranges for a call frequency metric to use for categorizing candidate oligonucleotide probes. For instance, a call frequency metric represents a value between 0 and 1 that indicates a percentage of genomic samples at each locus with call scores above a no-call-genotype-call-quality threshold (e.g., a threshold GenCall score below or equal to which there is no genotype call). In some cases, the probe design system 106 identifies (i) an upper limit of 0.99 for a call frequency metric above or equal to which a candidate oligonucleotide probe satisfies one threshold for the favorable probe-accuracy-training class 308 and (ii) a lower limit of 0.97 for a call frequency metric below or equal to which a candidate oligonucleotide probe satisfies one threshold for the unfavorable probe-accuracy-training class 310.

As also indicated above, the probe design system 106 can identify threshold ranges for intensity-value metrics to use for categorizing candidate oligonucleotide probes. For instance, the probe design system 106 identifies threshold ranges for average R intensity values indicating an average normalized intensity value of a light signal (e.g., emitted by an oligonucleotide probe's label). In particular, average R intensity values can represent an average of AA, AB, and BB R Means for intensity values corresponding to AA clusters of intensity values for a first allele, AB clusters of intensity values for a first and second allele, and BB clusters of intensity values for the second allele. In some cases, the probe design system 106 identifies (i) an upper limit of 0.4 for average R intensity values above or equal to which a candidate oligonucleotide probe satisfies one threshold for the favorable probe-accuracy-training class 308 and (ii) a lower limit of 0.2 for average R intensity values below or equal to which a candidate oligonucleotide probe satisfies one threshold for the unfavorable probe-accuracy-training class 310.

As a further example of a threshold range for an intensity-value metric, in some embodiments, the probe design system 106 identifies threshold ranges for a cluster separation score associated with a candidate oligonucleotide probe. A cluster separation score measures distances among genotype clusters along a theta dimension. In particular, in some cases, a cluster separation score ranges from 0 to 1 and measures distance between the closest genotype clusters for a microarray. In certain embodiments, the probe design system 106 identifies (i) an upper limit of 0.6 for a cluster separation score above or equal to which a candidate oligonucleotide probe satisfies one threshold for the favorable probe-accuracy-training class 308 and (ii) a lower limit of 0.3 for a cluster separation score below or equal to which a candidate oligonucleotide probe satisfies one threshold for the unfavorable probe-accuracy-training class 310.

As suggested above, the probe design system 106 can also identify threshold ranges for certain error metrics (e.g., inheritance error metrics and reproducibility error metrics) to use for categorizing candidate oligonucleotide probes. In some cases, an error metric combines different errors, such as an error combination metric that combines a number of one or more PC errors, PPC errors, or reproducibility errors. For instance, in some embodiments, the probe design system 106 identifies threshold ranges for a combination of PC errors, PPC errors, and reproducibility errors associated with an oligonucleotide probe.

As the name indicates, parent-child (PC) error represents a parent-child error where the child genomic sample is given a genotype call that is an impossible genotype given a parent's genotype. Similarly, a parent-parent-child (PPC) error represents a parent-parent-child errors where the child sample is given a genotype call that is an impossible genotype given both parents' genotypes. Both PC and PPC errors accordingly measure deviations from expected allelic inheritance patterns (e.g., Mendelian inheritance patterns) in matched parent and child genomic samples. PC and PPC values range from 0 to three times maximum number of trios. By contrast, reproducibility errors measure a reproducibility of genotype calls for replicate genomic samples at each genomic coordinate corresponding to an SNP or other variant. Reproducibility errors include values ranging from 0 to maximum a number of replicates.

In some cases, the probe design system 106 identifies (i) an upper limit of 2 combined PC-PPC-Reproducibility errors below or equal to which a candidate oligonucleotide probe satisfies one threshold for the favorable probe-accuracy-training class 308 and (ii) a lower limit of 5 combined PC-PPC-Reproducibility errors above or equal to which a candidate oligonucleotide probe satisfies one threshold for the unfavorable probe-accuracy-training class 310. As indicated above, however, the probe design system 106 can use any combination of or individual PC, PPC, and reproducibility errors for threshold ranges.

To summarize the example threshold ranges for the genotyping metrics described above, in some embodiments, the probe design system 106 identifies the upper limits of genotyping metrics for categorizing candidate oligonucleotide probes into the favorable probe-accuracy-training class 308 and lower limits of genotyping metrics for categorizing candidate oligonucleotide probes into the unfavorable probe-accuracy-training class 310—as set forth in Table 1 below.

TABLE 1

| Genotyping Metric | Lower Limit | Upper Limit |
|---|---|---|
| GenTrain Score | 0.3 | 0.85 |
| Call Frequency Metric | 0.97 | 0.99 |
| Average R (Intensity) | 0.2 | 0.4 |
| PC + PPC + Rep Errors | 5 | 2 |
| Cluster Separation Score | 0.3 | 0.6 |
| 10% GenCall Score | 0.3 | 0.6 |
| NextGen1 Score | 0.7 | 0.7 |

As further shown in FIG. 3A, the probe design system 106 categorizes the candidate oligonucleotide probes 302a-302i into either the favorable probe-accuracy-training class 308 or the unfavorable probe-accuracy-training class 310 based on one or more threshold ranges of the genotyping metrics 304. For instance, in some embodiments, the probe design system 106 categorizes the candidate oligonucleotide probes 302a-302i into either the favorable probe-accuracy-training class 308 or the unfavorable probe-accuracy-training class 310 based on upper limits and lower limits of some or all genotyping metrics, such as one or more of genotype-call-quality metrics, call frequency metrics, intensity-value metrics, or error metrics (e.g., inheritance error metrics and/or reproducibility error metrics).

In one particular embodiment, for instance, based on upper limits and lower limits of GenTrain score and average normalized intensity value, the probe design system 106 categorizes the candidate oligonucleotide probes 302a-302i into either the favorable probe-accuracy-training class 308 or the unfavorable probe-accuracy-training class 310. As shown in FIG. 3A, based on the threshold ranges set forth in Table 1, the probe design system 106 categorizes the candidate oligonucleotide probes 302a, 302b, 302c, and 302d into either the favorable probe-accuracy-training class 308 and the candidate oligonucleotide probes 302g, 302h, and 302i into the unfavorable probe-accuracy-training class 310. Because the threshold ranges for genotyping metrics in Table 1 are relatively numerous, embodiments of the probe design system 106 that utilize each of the threshold ranges for genotyping metrics in Table 1 tend to categorize fewer candidate oligonucleotide probes into the favorable probe-accuracy-training class 308 than embodiments utilizing threshold ranges for fewer genotyping metrics.

As further indicated by FIG. 3A, in some cases, the probe design system 106 cannot classify candidate oligonucleotide probes into either the favorable probe-accuracy-training class 308 or the unfavorable probe-accuracy-training class 310 based on upper limits and lower limits of the genotyping metrics 304. In such cases, the probe design system 106 identifies such candidate oligonucleotide probes as indeterminate in terms of probe accuracy (e.g., genotyping accuracy or binding accuracy) and optionally classifies such candidate oligonucleotide probes into an indeterminate probe-accuracy-training class 312. Based on the threshold ranges set forth in Table 1, for example, the probe design system 106 categorizes the candidate oligonucleotide probes 302e and 302f into the indeterminate probe-accuracy-training class 312.

As indicated above, FIG. 3B illustrates the probe design system 106 categorizing training candidate oligonucleotide probes for existing microarrays into favorable and unfavorable probe-accuracy-training classes for training a probe-classification-machine-learning model. In particular, the probe design system 106 categorizes training candidate oligonucleotide probes with genotyping metrics from a Global Screen Array (GSA) microarray 314 and a Global Diversity Array (GDA) microarray 316 into favorable and unfavorable probe-accuracy-training classes based on genotyping-metric threshold ranges.

As shown in table 318, for instance, the probe design system 106 identifies candidate oligonucleotide probes with genotyping metrics from both the GDA microarray 314 and GSA microarray 316 as part of classifying training candidate oligonucleotide probes. In some cases, the probe design system 106 applies a GenTrain algorithm to determine clusters of intensity values corresponding to the candidate oligonucleotide probes and categorizes into the unfavorable probe-accuracy-training class 310 (or discards) a subset of candidate oligonucleotide probes that fail to satisfy one or more threshold genotyping metrics. In some cases, based on a GenTrain score or other metrics for clusters of intensity values from the GenTrain algorithm, researchers perform a quality check and discard (or categorize into the unfavorable probe-accuracy-training class 310) certain candidate oligonucleotide probes that are redundant of other candidate oligonucleotide probes or that fail to satisfy one or more threshold genotyping metrics. As an example of using such threshold genotyping metrics, in some embodiments, the probe design system 106 (or researchers) discard or categorize into the unfavorable probe-accuracy-training class 310 a subset of candidate oligonucleotide probes that fail to satisfy one or more of a threshold GenTrain score, a threshold cluster separation score, or a threshold call frequency metric.

Regardless of whether candidate oligonucleotide probes are discarded or pre-categorized into the unfavorable probe-accuracy-training class 310, as indicated above, training candidate oligonucleotide probes can be categorized or classified based on threshold ranges of one or more of genotyping metrics for the purposes of training a probe-classification-machine-learning model. In certain implementations, for instance, the probe design system 106 categorizes (i) into the unfavorable probe-accuracy-training class 310 a first subset of candidate oligonucleotide probes that satisfy one or more lower limits of genotyping metrics set forth in Table 2 below and (ii) into the indeterminate probe-accuracy-training class 312 a second subset of candidate oligonucleotide probes that satisfy one or more limits of genotyping metrics set forth in Table 2 below:

TABLE 2

| | Lower Limits for | Limits for |
|---|---|---|
| Genotyping Metric | Unfavorable | Indeterminate |
| GenTrain Score | <0.3 | <0.4 |
| Concordance | <0.99 | N/A |
| Call Frequency Metric | <0.97 | ≤0.99 |
| AA R Mean | ≤0.2 | ≤0.4 |
| AB R Mean | ≤0.2 | ≤0.4 |
| BB R Mean | ≤0.2 | ≤0.4 |
| A/B Frequency | N/A | ≤0.4 |
| AB T Mean | N/A | (<0.2) or (>0.8) |
| Average R (Intensity) | <0.2 | N/A |
| Reproducibility Errors | >2 | >0 |
| PPC Errors | >2 | >0 |
| PC + PPC + Rep Errors | >5 | N/A |
| Cluster Separation Score | ≤0.3 | <0.45 |
| 10% GenCall Score | N/A | ≤0.3 |
| NextGen1 Score | N/A | <0.7 |
| Hex Excess | N/A | >0.2 |

To apply the GenTrain algorithm, in some cases, the probe design system 106 generally measures intensity values emitted by the candidate oligonucleotide probes (bound to target nucleotides) from both the GDA microarray 314 and GSA microarray 316. The probe design system 106 subsequently clusters the intensity values according to different clustering models and selects a clustering model that best fits the clusters of intensity values. The probe design system 106 can determine GenTrain scores for the candidate oligonucleotide probes both before and after applying the GenTrain algorithm.

As table 318 in FIG. 3B indicates, before applying a GenTrain algorithm, the GSA microarray 316 includes approximately 684,543 candidate oligonucleotide probes, and the GDA microarray 314 includes approximately 2,236,241 candidate oligonucleotide probes—with 555,686 candidate oligonucleotide probes shared between the GSA microarray 316 and the GDA microarray 314. After applying a GenTrain algorithm and removing probes that fail to satisfy one or more threshold genotyping metrics (e.g., GenTrain score), the GSA microarray 316 includes approximately 642,031 candidate oligonucleotide probes, and the GDA microarray 314 includes approximately 1,906,815 candidate oligonucleotide probes—with 464,323 candidate oligonucleotide probes shared between the GSA microarray 316 and the GDA microarray 314.

As further shown in FIG. 3B, graphs 320a and 320b depict candidate oligonucleotide probes from the GSA microarray 316 and the GDA microarray 314, respectively, according to GenTrain scores along both vertical and horizontal axes. As shown by the graph 320a, for instance, most of the candidate oligonucleotide probes from the GSA microarray 316 and the GDA microarray 314—before the GenTrain algorithm is applied—are grouped together based on GenTrain score with a correlation of 0.72. As shown by the graph 320b, by contrast, more of the candidate oligonucleotide probes from the GSA microarray 316 and the GDA microarray 314—after the GenTrain algorithm is applied and quality control applied to remove a subset of candidate oligonucleotide probes—are grouped together based on GenTrain score with a higher correlation of 0.94.

As further shown in FIG. 3B, the probe design system 106 categorizes the candidate oligonucleotide probes from the GSA microarray 316 and the GDA microarray 314 into either the favorable probe-accuracy-training class 308 or the unfavorable probe-accuracy-training class 310— based on a combination of threshold ranges of genotyping metrics. For instance, in some embodiments, the probe design system 106 categorizes approximately 2,073,394 candidate oligonucleotide probes from the GSA microarray 316 and the GDA microarray 314 into the favorable probe-accuracy-training class 308 and approximately 305,529 candidate oligonucleotide probes from the GSA microarray 316 and the GDA microarray 314 into the unfavorable probe-accuracy-training class 310 based on the upper limits and lower limits for genotyping metrics set forth in Table 1 above. Having categorized candidate oligonucleotide probes into favorable and unfavorable probe-accuracy-training classes, the probe design system 106 develops ground-truth classifications for the candidate oligonucleotide probes to train a probe-classification-machine-learning model.

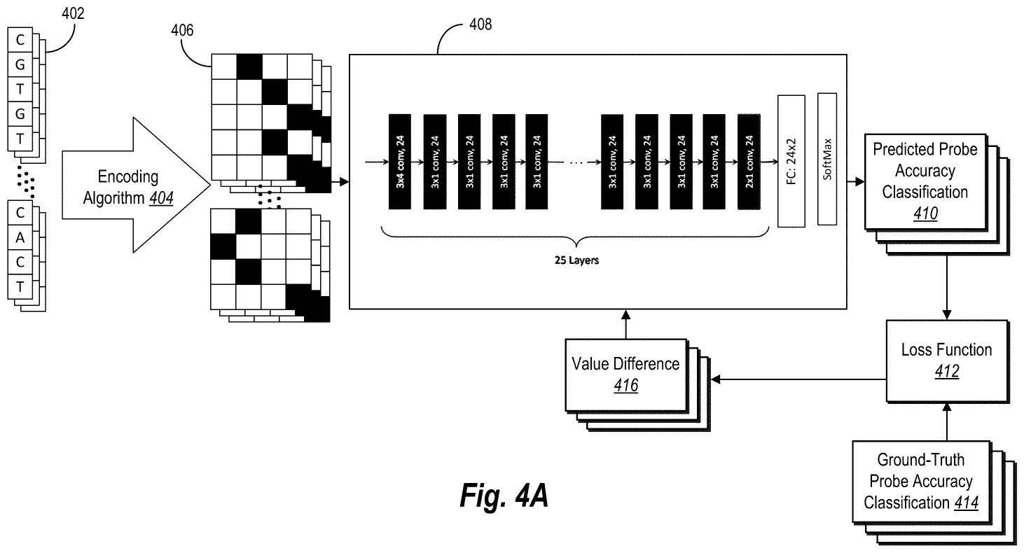

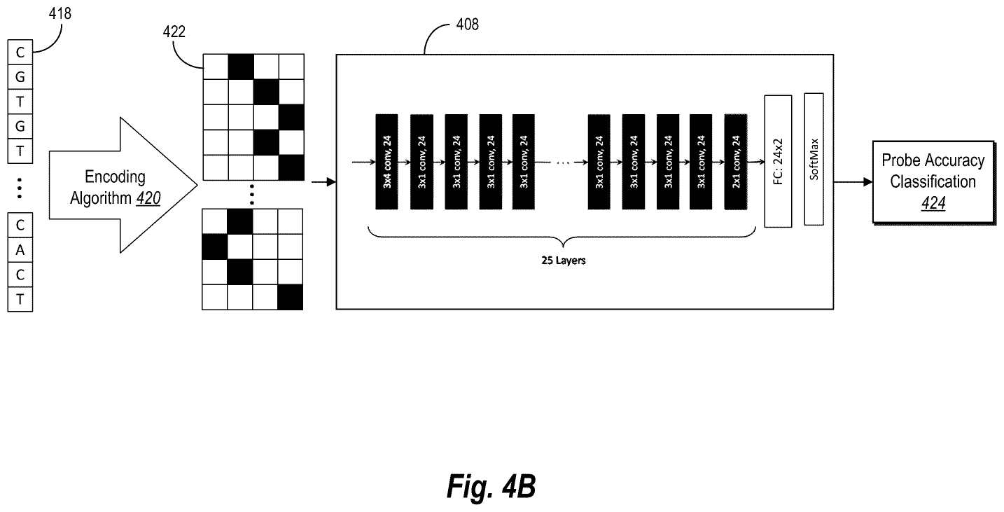

As just suggested, in some embodiments, the probe design system 106 trains a probe-classification-machine-learning model to determine probe accuracy classifications specific to the nucleotide-sequence composition of oligonucleotide probes. In accordance with one or more embodiments, FIG. 4A depicts the probe design system 106 training a probe-classification-machine-learning model 408 to determine probe accuracy classifications that reflect a genotyping accuracy or a binding accuracy of a given oligonucleotide probe according to a specific nucleotide-sequence composition of the given oligonucleotide probe.

For simplicity, this disclosure describes an initial training iteration of the probe-classification-machine-learning model 408 followed by a summary of subsequent training iterations depicted in FIG. 4A. In an initial training iteration depicted by FIG. 4A, for example, the probe design system 106 inputs into the probe-classification-machine-learning model 408 a training dataset 406 representing a nucleotide sequence 402 of a candidate oligonucleotide probe. As indicated above, the nucleotide sequence 402 of the candidate oligonucleotide probe may comprise various numbers of nucleobases, such as a nucleotide sequencing spanning 30 to 150 nucleobases in length. Further, the training dataset 406 may take the form of a training feature vector, a training feature map, or a training matrix.

To process the nucleotide sequence 402 of the candidate oligonucleotide probe, in some embodiments, the probe design system 106 performs an encoding algorithm 404 to transform the nucleotide sequence 402 of the candidate oligonucleotide probe from nucleobases (or letters representing nucleobases) into the training dataset 406 representing the nucleotide sequence 402. For instance, the probe design system 106 can perform one-hot coding as the encoding algorithm 404 to transform the letters to a training feature map as the training dataset 406. In the alternative to one-hot coding, the probe design system 106 can use any suitable encoding algorithm, such as a target encoding algorithm or a leave-one-out encoding algorithm. The probe design system 106 further inputs the training dataset 406 representing the nucleotide sequence 402 into the probe-classification-machine-learning model 408.

In some cases, the probe design system 106 encodes subsets of candidate oligonucleotide probes corresponding to one or both of a first allele and a second allele to train the probe-classification-machine-learning model 408. When encoding oligonucleotide probes corresponding to an Infinium® microarray from Illumina, Inc., in some embodiments, the probe design system 106 one-hot encodes a first allele oligonucleotide probes that correspond to a first allele and comprise fifty nucleobases. Similarly, the probe design system 106 one-hot encodes a second allele oligonucleotide probes that correspond to a second allele and comprise fifty nucleobases. Accordingly, the probe design system 106 can encode oligonucleotide probes corresponding to a first allele or a second allele for training.

To test whether using oligonucleotide probes for either allele influenced training accuracy for the probe-classification-machine-learning model 408, researchers tested an impact of using different subsets of candidate oligonucleotide probes from the Infinium microarray on true-positive rate and true-negative rate of determining probe accuracy classifications. In addition to one-hot encoding a first allele and a second allele oligonucleotide probes corresponding to first and second alleles, in some embodiments, the probe design system 106 (i) one-hot encoded the initial forty-nine nucleobases of nucleotide sequences from first allele oligonucleotide probes and determined a union of one-hot encoded last nucleobase of from both the first and second allele oligonucleotide probe and (ii) one-hot encoded the initial forty-nine nucleobases of nucleotide sequences from first allele oligonucleotide probes and determined an intersection of one-hot encoded last nucleobase of from both the first and second allele oligonucleotide probe. As shown by Table 3 below, the encoding approach did not appear to affect the true-positive rate and true-negative rate of the probe-classification-machine-learning model 408 determining probe accuracy classifications.

| Encoded Probe Type | True-Positive Rate | True-Negative Rate |
|---|---|---|
| First Allele Oligonucleotide | 0.78 | 0.63 |
| Probes | | |
| Second Set of Oligonucleotide | 0.76 | 0.65 |
| Probes | | |
| Union | 0.77 | 0.65 |
| Intersection | 0.78 | 0.64 |

As further indicated above, in some embodiments, the probe-classification-machine-learning model 408 exhibits a unique architecture that comprises layers customized to detect motifs or other nucleotide-sequence patterns. As depicted in FIG. 4A, for instance, a CNN with a customized architecture constitutes the probe-classification-machine-learning model 408. In particular, the customized CNN comprises approximately twenty-five convolutional layers each with twenty-four channels—where most of the convolutional layers include a convolutional kernel size of three—followed by a fully connected layer and a SoftMax layer. By using a kernel size of three that can analyze a window of data representing three nucleobases from an oligonucleotide probe's nucleotide sequence at a time, the probe-classification-machine-learning model 408 includes a matrix customized for recognizing motifs or other nucleotide-sequence patterns and determine corresponding dot products.

As further depicted in FIG. 4A, the customized CNN comprises convolutional layers with twenty-four channels that have been selected through experimentation to avoid overfitting the trained CNN to a training dataset and avoid computational complexity that might come with more channels (e.g., sixty-four channels)—while maintaining accurate predicted probe accuracy classifications. By using convolutional layers with twenty-four channels, the channels of the convolutional layers can retain feature representations of different nucleobase classes (e.g., A, T, C, G).

While the probe-classification-machine-learning model 408 depicted in FIG. 4A includes a certain number of convolutional layers for a nucleotide sequence of a certain number of nucleobases for an oligonucleotide probe, the probe design system 106 may adjust the number of convolutional layers and other layer parameters of a CNN for a different length of nucleotide sequence. For instance, in certain implementations, the probe-classification-machine-learning model 408 includes a kernel size of four, five, or more to facilitate analyzing longer motifs. In some cases, the probe design system 106 uses batch normalization and ReLu activations respectively before and after each convolutional layer. As suggested above, a different CNN or different neural network may likewise be used as the probe-classification-machine-learning model 408 with different layers.

In some cases, a CNN, such as that depicted in FIG. 4A, outperforms other deep neural networks because the input patterns for a nucleotide sequence are local and the input datasets are relatively smaller in size—without a need to capture long range dependencies. As shown in the customized CNN of FIG. 4A, the probe-classification-machine-learning model 408 lacks pooling layers to ensure that the CNN includes layers that detect and recognize location sensitivity for a nucleotide sequence of different nucleobase classes (e.g., A, T, C, G). To compensate for a lack of pooling layers and increase receptivity through the CNN, the probe-classification-machine-learning model 408 depicted in FIG. 4A includes un-padded convolutional kernels that gradually reduce a length of the feature map as the training dataset 406.

As indicated above, in some embodiments, a probe-classification-machine-learning model can take the form of a different architecture. In certain cases, for instance, a probe-classification-machine-learning model takes the form of a random-forest model or other series of decision trees performing a regression analysis. When using a series of decision trees as a regressor, the probe design system 106 can employ the series of decision trees to execute a regression. For instance, the probe-classification-machine-learning model can include decision trees that each include different decision nodes and determine preliminary probe accuracy classifications. The probe design system 106 can accordingly train various decision nodes within the decision trees to correctly determine in the aggregate a score or other probe accuracy classification indicating a degree to which a given oligonucleotide probe of a particular nucleotide sequence (i) yields an accurate or an inaccurate genotype call or (ii) accurately or inaccurately binds to a target oligonucleotide for genotyping.

As depicted in FIG. 4A, the probe-classification-machine-learning model 408 generates a predicted probe accuracy classification 410 based on the training dataset 406 representing the nucleotide sequence 402 of a candidate oligonucleotide probe. As indicated above, in some embodiments, the predicted probe accuracy classification 410 indicates a probability or likelihood that the candidate oligonucleotide probe (i) yields an accurate or an inaccurate genotype call for a target oligonucleotide or (ii) accurately or inaccurately binds to or hybridizes with the target oligonucleotide for genotyping.

As further shown in FIG. 4A, after determining the predicted probe accuracy classification 410, the probe design system 106 compares the predicted probe accuracy classification 410 to a ground-truth probe accuracy classification 414 for the nucleotide sequence 402 of the candidate oligonucleotide probe. In some cases, the ground-truth probe accuracy classification 414 represents a score between 0 and 1 in terms of a probability. In other cases, the ground-truth probe accuracy classification 414 represents a classification from a binary, ternary, quaternary, or other multi-part probe accuracy classification scheme.

In some implementations, the probe design system 106 uses a loss function 412 to compare (and determine any difference) between the predicted probe accuracy classification 410 and the ground-truth probe accuracy classification 414. As shown in FIG. 4A, the probe design system 106 determines a value difference 416 (e.g., a loss) between the predicted probe accuracy classification 410 and the ground-truth probe accuracy classification 414 utilizing the loss function 412. In certain embodiments, for instance, the probe design system 106 uses a cross-entropy loss function for a CNN, where a loss is determined by comparing (i) a vector of probabilities for different probe accuracy classifications (e.g., [0.7, 0.3]) from a SoftMax layer and (ii) a vector of ground-truth values from the ground-truth probe accuracy classification (e.g., [1, 0]).

Depending on the form of the probe-classification-machine-learning model 408, the probe design system 106 can use one or a variety of loss functions for the loss function 412. In certain embodiments, for instance, the probe design system 106 uses a means square error (MSE) function for a CNN, where a loss is determined using a value of 1 representing the ground-truth probe accuracy classification 414 and a value between 0 and 1 representing the predicted probe accuracy classification 410. In contrast, in some embodiments, the probe design system 106 uses a logistic loss function (e.g., for a logistic regression model) or a least-squared-error function (e.g., for a LSTM).

Based on the determined value difference 416 from the loss function 412, the probe design system 106 modifies parameters (e.g., network parameters) of the probe-classification-machine-learning model 408. By adjusting the parameters over training iterations, the probe design system 106 increases the accuracy with which the probe-classification-machine-learning model 408 determines predicted probe accuracy classifications. Based on the determined value difference 416, for instance, the probe design system 106 determines a gradient for weights using stochastic gradient descent (SGD). In some cases, the probe design system 106 uses the following function: w:=w−η∇Q(w)=w−η/nΣᵢ₌₁ⁿ∇Qᵢ(w), where w represents a weight of the probe-classification-machine-learning model 408 and ΔQᵢ represents a gradient. After determining the gradient, the probe design system 106 adjusts weights of the probe-classification-machine-learning model 408 based on the gradient in a given training iteration. In the alternative to SGD, the probe design system 106 can use gradient descent or a different optimization method for training across training iterations.

After the initial training iteration and parameter modification, as further indicated by FIG. 4A, the probe design system 106 further determines predicted probe accuracy classifications for training datasets representing different nucleotide sequences for different candidate oligonucleotide probes. In some cases, the probe design system 106 performs training iterations until the parameters (e.g., value or weights) of the probe-classification-machine-learning model 408 do not change significantly across training iterations or otherwise satisfy a convergence criteria.

In addition to training the probe-classification-machine-learning model 408, in some embodiments, the probe design system 106 implements a trained version of the probe-classification-machine-learning model 408. In accordance with one or more embodiments, FIG. 4B depicts the probe design system 106 using the probe-classification-machine-learning model 408 to determine a probe accuracy classification for an oligonucleotide probe based on the oligonucleotide probe's nucleotide-sequence composition. As indicated above, the probe design system 106 can determine probe accuracy classifications for one or more oligonucleotide probes from a set of candidate oligonucleotide probes.

As shown by FIG. 4B, for example, the probe design system 106 inputs into a trained version of the probe-classification-machine-learning model 408 a dataset 422 representing a nucleotide sequence 418 of an oligonucleotide probe. As indicated above, the nucleotide sequence 418 of the oligonucleotide probe may comprise various numbers of nucleobases, such as a nucleotide sequencing spanning 30 to 150 nucleobases in length. Further, the dataset 422 may take the form of a feature vector, a feature map, or a matrix. As shown in FIG. 4B, the dataset 422 takes the form of a feature map.

To process the nucleotide sequence 418 of the oligonucleotide probe, in some embodiments, the probe design system 106 performs an encoding algorithm 420 to transform the nucleotide sequence 418 of the oligonucleotide probe from nucleobases (or letters representing nucleobases) into the dataset 422 representing the nucleotide sequence 418. For instance, the probe design system 106 can perform one-hot coding as the encoding algorithm 420 to transform the letters to a training feature map as the dataset 422. In the alternative to one-hot coding, the probe design system 106 can use any suitable encoding algorithm, such as a target encoding algorithm or a leave-one-out encoding algorithm.

As further shown in FIG. 4B, the probe design system 106 further inputs the dataset 422 representing the nucleotide sequence 418 into a trained version of the probe-classification-machine-learning model 408. As depicted in FIG. 4B and consistent with the disclosure above, the probe-classification-machine-learning model 408 generates a probe accuracy classification 424 based on the dataset 422 representing the nucleotide sequence 418 of the oligonucleotide probe. In some embodiments, for instance, the probe-classification-machine-learning model 408 generates scores representing different probabilities of different probe accuracy classifications. For instance, the probe-classification-machine-learning model 408 generates a vector of probabilities for a favorable probe accuracy classification and an unfavorable probe accuracy classification (e.g., [0.7, 0.3]). In other cases, the probe-classification-machine-learning model 408 generates a vector of probabilities for binary, ternary, quaternary, or other multi-part probe accuracy classification schemes. In addition, or in the alternative, the probe-classification-machine-learning model 408 generates (or the probe design system 106 converts probabilities into) a textual label as a probe accuracy classification from a binary (e.g., 0 or 1), ternary (e.g., 1, 2, or 3), quaternary (e.g., 1, 2, 3, or 4), or other multi-part probe accuracy classification scheme.

In the alternative from outputs of a neural network, in some embodiments, a probe-classification-machine-learning model takes the form of a random-forest model or other series of decision trees. As suggested above, as a series of decision trees, the probe-classification-machine-learning model can include decision nodes that determine preliminary scores as preliminary probe accuracy classifications. After the decision trees generate the preliminary scores, in certain implementations, the probe-classification-machine-learning model performs a consensus operation on the preliminary scores to generate a final score (e.g., probability) as a probe accuracy classification for an oligonucleotide probe comprising a nucleotide sequence. For instance, in certain embodiments, the probe-classification-machine-learning model averages (or determines a weighted average of) the preliminary scores to generate the probe accuracy classification. By averaging or otherwise combining the preliminary scores, in some embodiments, a final score as the probe accuracy classification represents a more accurate value that avoids overfitting to training data by any individual decision tree.

While FIG. 4B depicts a single probe accuracy classification, the probe-classification-machine-learning model 408 has been trained to generate probe accuracy classifications for different oligonucleotide probes specific to different nucleotide-sequence compositions. Accordingly, the probe-classification-machine-learning model 408 can generate probe accuracy classifications specific to different nucleotide sequences input as encoded datasets.

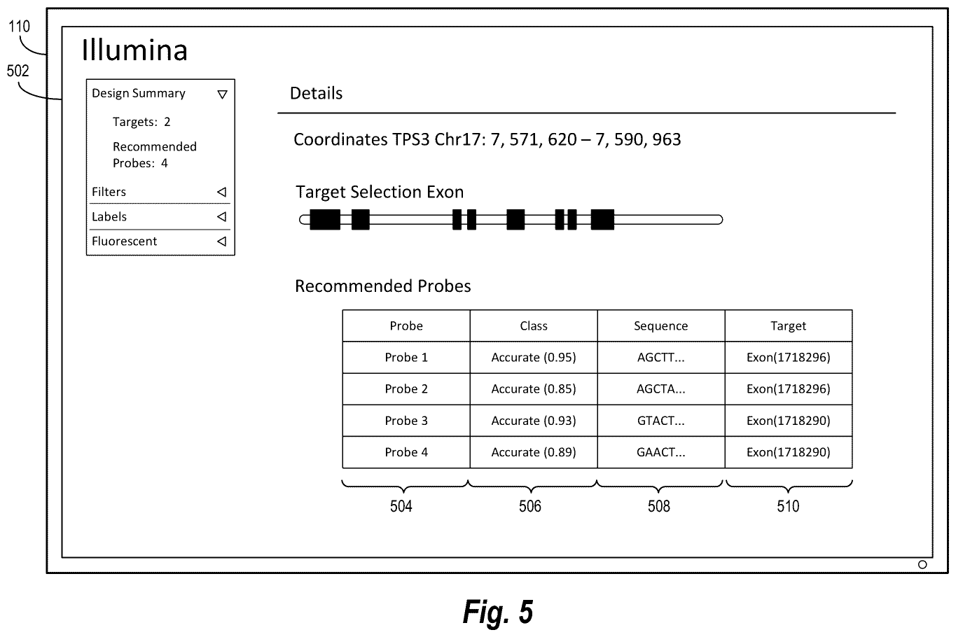

In addition to determining probe accuracy classifications, in some embodiments, the probe design system 106 recommends oligonucleotide probes for use in a microarray based on the probe accuracy classifications. In accordance with one or more embodiments, FIG. 5 depicts the user client device 110 presenting a graphical user interface 502 for a probe design application that suggests oligonucleotide probes for a microarray. While FIG. 5 depicts the graphical user interface 502 displayed when the user client device 110 implements computer-executable instructions of the microarray application 112, rather than repeatedly refer to the computer-executable instructions causing the user client device 110 to perform certain actions for the probe design system 106, this disclosure describes the user client device 110 or the probe design system 106 performing those actions in the following paragraphs.

As shown in FIG. 5, the user client device 110 presents, within the graphical user interface 502, recommended-probe identifiers 504 for recommended oligonucleotide probes and probe accuracy classifications 506 generated by a probe-classification-machine-learning model. Alongside or otherwise nearby each of the recommended-probe identifiers 504, the user client device 110 presents a probe accuracy classification from the probe accuracy classifications 506. For each of the probe accuracy classifications 506, for instance, the user client device 110 presents a textual label describing the corresponding probe accuracy classification and a score indicating a probability that the recommended oligonucleotide probe yields an accurate genotype call or accurately binds a target oligonucleotide. In some embodiments, however, the user client device 110 presents one of the textual label or the score, but not both.

In addition to the recommended-probe identifiers 504 and the probe accuracy classifications 506, the user client device 110 presents (i) nucleotide-sequence options 508 for nucleotide sequences of the recommended oligonucleotide probes and (ii) target-oligonucleotide identifiers 510 for target oligonucleotides corresponding to the recommended oligonucleotide probes. As shown in FIG. 5, each of the nucleotide-sequence options 508 includes letters or other indicators of part of the corresponding nucleotide sequence for the recommended oligonucleotide probes. Based on detecting a selection of a nucleotide-sequence option, in some embodiments, the user client device 110 presents letters representing a full nucleotide sequence of the recommended oligonucleotide probe. As further shown in FIG. 5, each of the target-oligonucleotide identifiers 510 includes a description or numerical code for the corresponding target oligonucleotide for a recommended oligonucleotide probe. Based on detecting a selection of a target-oligonucleotide identifier, in some embodiments, the user client device 110 presents one or more of genomic coordinates for the corresponding target oligonucleotide and a nucleotide sequence of the target oligonucleotide.

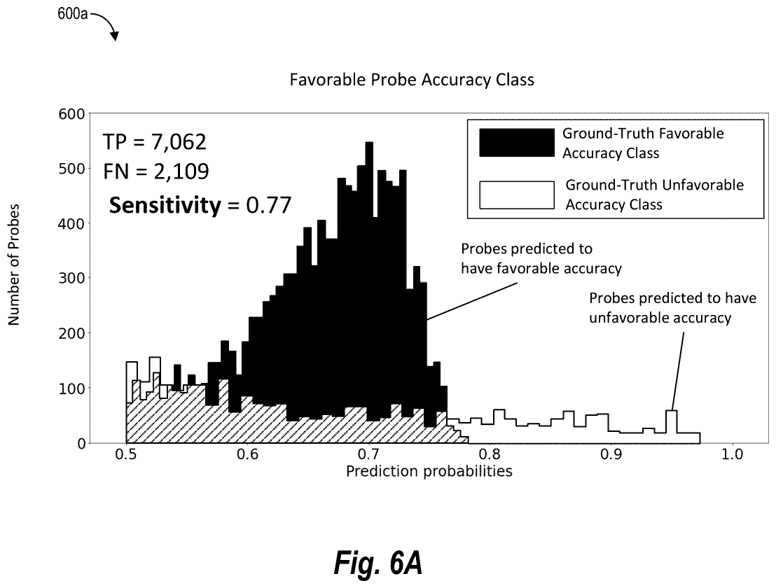

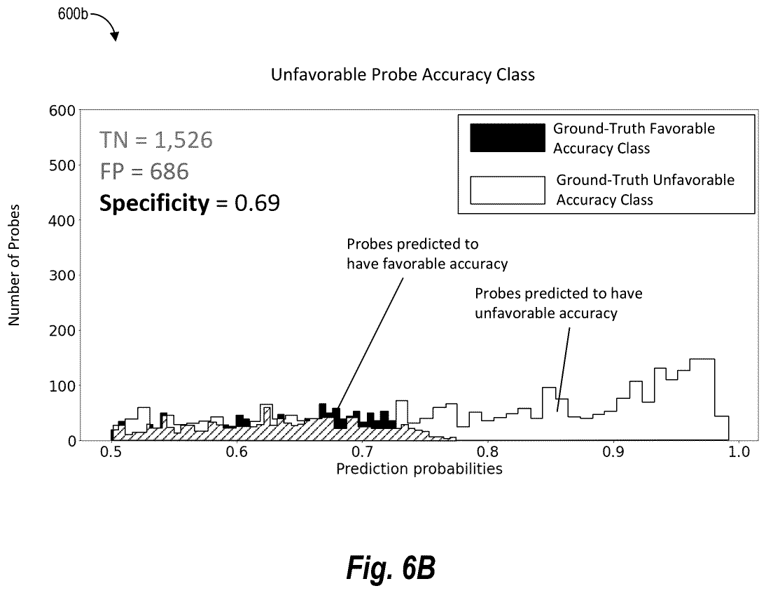

To test the accuracy of probe accuracy classifications by a probe-classification-machine-learning model, researchers applied a trained probe-classification-machine-learning model to a validation dataset of candidate oligonucleotide probes. In accordance with one or more embodiments, FIGS. 6A-6B depict graphs showing true-positive and false-negative rates of a trained version of the probe-classification-machine-learning model categorizing oligonucleotide probes into probe accuracy classifications.

As part of the validation, the researchers input approximately 28,755 candidate nucleotide probes from a Pharmacogenomic (PGx)-Global Diversity Array (GDA) microarray (hereinafter, PGx-GDA probes) available from Illumina, Inc. The PGx-GDA probes differ from the microarray probes used to train the same probe-classification-machine-learning model. The researchers further assigned each PGx-GDA probe to either a ground-truth favorable probe accuracy class or a ground-truth unfavorable probe accuracy class based on (i) a F-measure for the given PGx-GDA probe of accurately yielding a genotype call in a microarray, (ii) a number of evidence or instances of genotype calls to support a PGx variant call from Next Generation Sequencing (NGS) truth data, and (iii) a reference call frequency for a given target nucleotide corresponding to the given PGx-GDA probe.

In particular, the researchers assigned 14,094 PGx-GDA probes to a ground-truth favorable probe accuracy classification based on such probes exhibiting an F-measure of ≥98.5%, evidence of ≥10 PGx variant calls from the NGS truth data, and a call frequency of ≥0.3; 4,196 PGx-GDA probes to a ground-truth unfavorable probe accuracy classification based on such probes exhibiting an F-measure of <98.5% and a call frequency of ≥0.3; and 10,465 PGx-GDA probes to a ground-truth indeterminate probe accuracy classification based on such probes exhibiting an F-measure of <98.5%, evidence of <10 PGx variant calls from the NGS truth data, and a call frequency of <0.3.

Based on the 28,755 PGx-GDA probes, the trained version of the probe-classification-machine-learning model determined probe accuracy classifications. The researchers determined the true-positive rates and false-negative rates depicted in FIGS. 6A and 6B, respectively, based on the assigned ground-truth probe accuracy classifications for the 28,755 PGx-GDA probes.

As shown by a graph 600a in FIG. 6A, for example, the probe-classification-machine-learning model correctly determines favorable probe accuracy classifications for PGx-GDA probes with a sensitivity (or true-positive rate) of approximately 0.77 based on encoded datasets of the nucleotide sequences for the PGx-GDA probes. In particular, the probe-classification-machine-learning model (i) correctly determines favorable probe accuracy classifications for 7,062 PGx-GDA probes (as true positives) assigned ground-truth favorable probe accuracy classifications and (ii) incorrectly determines unfavorable probe accuracy classifications for 2,109 PGx-GDA probes (as false negatives) assigned ground-truth favorable probe accuracy classifications.

As shown by a graph 600b in FIG. 6B, by contrast, the probe-classification-machine-learning model correctly determines unfavorable probe accuracy classifications for PGx-GDA probes with a specificity (or true-negative rate) of approximately 0.69 on encoded datasets of the nucleotide sequences for the PGx-GDA probes. In particular, the probe-classification-machine-learning model (i) correctly determines unfavorable probe accuracy classifications for 1,526 PGx-GDA probes (as true negatives) assigned ground-truth unfavorable probe accuracy classifications and (ii) incorrectly determines favorable probe accuracy classifications for 686 PGx-GDA probes (as false positives) assigned ground-truth favorable probe accuracy classifications.

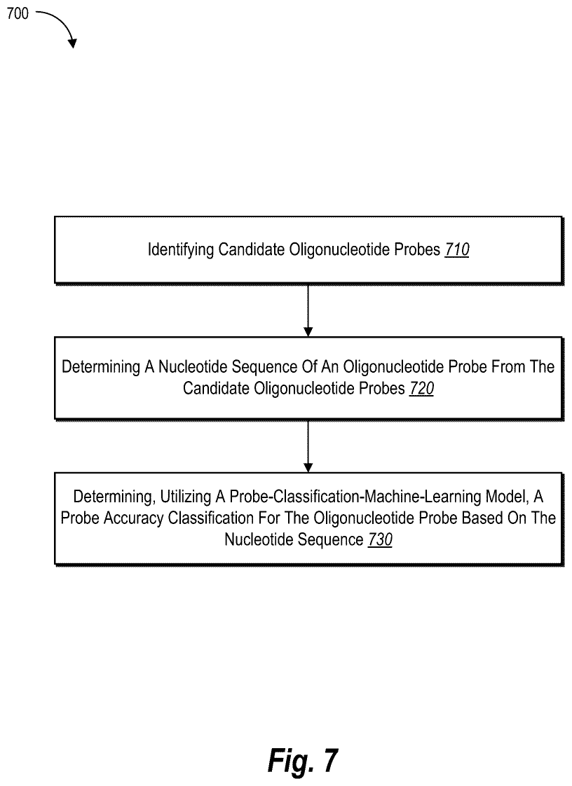

Turning now to FIG. 7, this figure illustrates a flowchart of a series of acts 700 of using a probe-classification-machine-learning model to determine probe accuracy classifications for oligonucleotide probes based on the oligonucleotide probes' nucleotide-sequence composition in accordance with one or more embodiments of the present disclosure. While FIG. 7 illustrates acts according to one embodiment, alternative embodiments may omit, add to, reorder, and/or modify any of the acts shown in FIG. 7. The acts of FIG. 7 can be performed as part of a method. Alternatively, a non-transitory computer readable storage medium can comprise instructions that, when executed by one or more processors, cause a computing device or a system to perform the acts depicted in FIG. 7. In still further embodiments, a system comprising at least one processor and a non-transitory computer readable medium comprising instructions that, when executed by one or more processors, cause the system to perform the acts of FIG. 7.

As shown in FIG. 7, the acts 700 include an act 710 of identifying candidate oligonucleotide probes. In particular, in some embodiments, the act 710 includes identifying candidate oligonucleotide probes for hybridizing with target oligonucleotides.

As further shown in FIG. 7, the acts 700 include an act 720 of determining a nucleotide sequence of an oligonucleotide probe from the candidate oligonucleotide probes. In particular, in some embodiments, the act 720 includes determining a nucleotide sequence of an oligonucleotide probe from the candidate oligonucleotide probes.

As suggested above, in certain embodiments, determining the probe accuracy classification comprises determining, for the oligonucleotide probe, a favorable genotyping accuracy class indicating a probability that the oligonucleotide probe yields an accurate genotype call or an unfavorable genotyping accuracy class indicating a probability that the oligonucleotide probe yields an inaccurate genotype call. Similarly, in certain implementations, determining the probe accuracy classification comprising determining, for the oligonucleotide probe and based on a dataset representing the nucleotide sequence, a favorable genotyping accuracy class indicating a probability that the oligonucleotide probe yields an accurate genotype call or an unfavorable genotyping accuracy class indicating a probability that the oligonucleotide probe yields an inaccurate genotype call.

As further suggested above, in some cases, determining the probe accuracy classification comprises determining, for the oligonucleotide probe, a favorable binding accuracy class indicating a probability that the oligonucleotide probe accurately binds to a target oligonucleotide for genotyping or an unfavorable binding accuracy class indicating a probability that the oligonucleotide probe inaccurately binds to the target oligonucleotide for genotyping. Relatedly, in certain embodiments, determining the probe accuracy classification comprises determining, for the oligonucleotide probe and based on a dataset representing the nucleotide sequence, a favorable binding accuracy class indicating a probability that the oligonucleotide probe accurately binds to a target oligonucleotide for genotyping or an unfavorable binding accuracy class indicating a probability that the oligonucleotide probe inaccurately binds to the target oligonucleotide for genotyping.

Further, in some cases, determining the probe accuracy classification comprises determining a score indicating a genotyping probability that the oligonucleotide probe yields an accurate genotype call or a binding probability that the oligonucleotide probe accurately binds to a target oligonucleotide for genotyping.

As further shown in FIG. 7, the acts 700 include an act 730 of determining, utilizing a probe-classification-machine-learning model, a probe accuracy classification for the oligonucleotide probe based on the nucleotide sequence. In particular, in certain implementations, the act 730 includes determining, utilizing a probe-classification-machine-learning model, a probe accuracy classification for the oligonucleotide probe based on the nucleotide sequence of the oligonucleotide probe. In some cases, the probe-classification-machine-learning model comprises a neural network or one or more decision trees.

In addition to the acts 710-730, in certain implementations, the acts 700 further include selecting the oligonucleotide probe for use in a microarray based on the probe accuracy classification. Relatedly, in certain cases, the acts 700 include hybridizing, utilizing a microarray, one or more copies of the oligonucleotide probe with one or more copies of a target oligonucleotide from a genomic sample; and determining a variant call for the genomic sample based on one or more copies of the oligonucleotide probe hybridizing with one or more copies of the target oligonucleotide. Similarly, in some embodiments, the acts 700 further include hybridizing, utilizing a microarray, one or more copies of the oligonucleotide probe with one or more copies of a target oligonucleotide corresponding to one or more genomic coordinates for a promoter region or a gene from a genomic sample; and determining a variant call for the one or more genomic coordinates of the genomic sample based on one or more copies of the oligonucleotide probe hybridizing with one or more copies of the target oligonucleotide.

As suggested above, in addition or in the alternative, in some embodiments, the acts 700 include determining, by the probe-classification-machine-learning model, feature values representing a pattern within the nucleotide sequence of the oligonucleotide probe corresponding to complimentary nucleobase bonds between the oligonucleotide probe and a target oligonucleotide; and determining, by the probe-classification-machine-learning model, the probe accuracy classification for the oligonucleotide probe based on the feature values representing the pattern. Relatedly, in some embodiments, determining the feature values representing the pattern within the nucleotide sequence comprises utilizing one or more filters of a kernel size customized for nucleotide-sequence-pattern recognition to determine the feature values representing the pattern.

Additionally, or alternatively, in certain implementations, the acts 700 further include determining, by the probe-classification-machine-learning model, feature values corresponding to nucleobases of one or more nucleobase classes within the nucleotide sequence of the oligonucleotide probe utilizing one or more channels customized for nucleobase-class recognition; and determining the probe accuracy classification for the oligonucleotide probe based on the feature values corresponding to the nucleobases of one or more nucleobase classes.

Further, in some cases, the acts 700 further include determining a different nucleotide sequence of an additional oligonucleotide probe from the candidate oligonucleotide probes; and determining, utilizing the probe-classification-machine-learning model, a different probe accuracy classification for the additional oligonucleotide probe based on the different nucleotide sequence of the additional oligonucleotide probe.

As noted above, the probe design system 106 can train a probe-classification-machine-learning model. In certain implementations, the acts 700 further include identifying threshold ranges for genotyping metrics indicating accurate probes and inaccurate probes for genotyping; and categorizing, based on the threshold ranges for genotyping metrics, the candidate oligonucleotide probes into a favorable probe-accuracy-training class for training the probe-classification-machine-learning model and an unfavorable probe-accuracy-training class for training the probe-classification-machine-learning model.

Relatedly for training, in some embodiments, the acts 700 further include identifying, from among the favorable probe-accuracy-training class or the unfavorable probe-accuracy-training class, a ground-truth oligonucleotide probe corresponding to the oligonucleotide probe; determining a value difference between a ground-truth probe accuracy classification for the ground-truth oligonucleotide probe and the probe accuracy classification for the oligonucleotide probe; and modifying one or more network parameters of the probe-classification-machine-learning model based on the value difference.

Further, in some embodiments, the acts 700 include selecting the oligonucleotide probe for use in a microarray based on the probe accuracy classification for the oligonucleotide probe; and presenting, for display within a graphical user interface of the computing device, a representation of the oligonucleotide probe as part of a recommended set of oligonucleotide probes for use in the microarray.

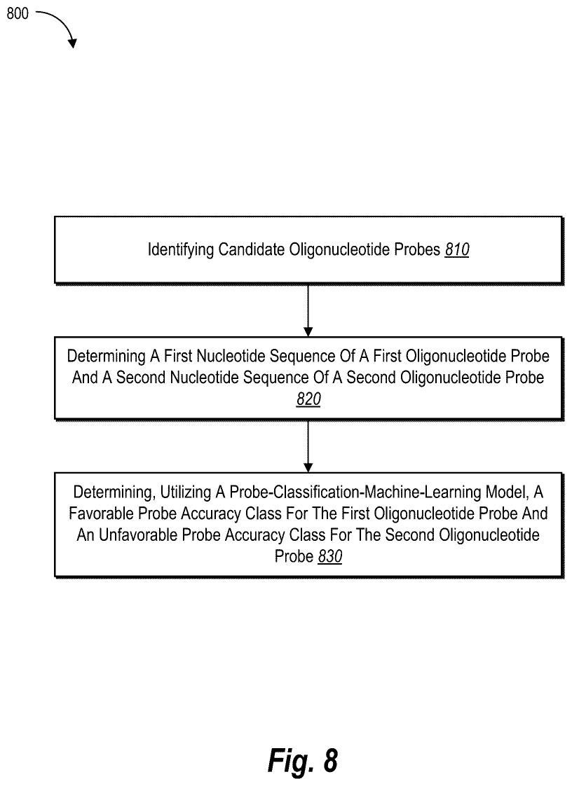

Turning now to FIG. 8, this figure illustrates a flowchart of a series of acts 800 of using a probe-classification-machine-learning model to determine favorable or unfavorable probe accuracy classifications for oligonucleotide probes based on the oligonucleotide probes' nucleotide-sequence composition in accordance with one or more embodiments of the present disclosure. While FIG. 8 illustrates acts according to one embodiment, alternative embodiments may omit, add to, reorder, and/or modify any of the acts shown in FIG. 8. The acts of FIG. 8 can be performed as part of a method. Alternatively, a non-transitory computer readable storage medium can comprise instructions that, when executed by one or more processors, cause a computing device or a system to perform the acts depicted in FIG. 8. In still further embodiments, a system comprising at least one processor and a non-transitory computer readable medium comprising instructions that, when executed by one or more processors, cause the system to perform the acts of FIG. 8.

As shown in FIG. 8, the acts 800 include an act 810 of identifying candidate oligonucleotide probes. In particular, in some embodiments, the act 810 includes identify candidate oligonucleotide probes for hybridizing with target oligonucleotides.

As further shown in FIG. 8, the acts 800 include an act 820 of determining a first nucleotide sequence of a first oligonucleotide probe and a second nucleotide sequence of a second oligonucleotide probe. In particular, in some embodiments, the act 820 includes determining a first nucleotide sequence of a first oligonucleotide probe from the candidate oligonucleotide probes and a second nucleotide sequence of a second oligonucleotide probe from the candidate oligonucleotide probes.

As further shown in FIG. 8, the acts 800 include an act 830 of determining, utilizing a probe-classification-machine-learning model, a favorable probe accuracy class for the first oligonucleotide probe and an unfavorable probe accuracy class for the second oligonucleotide probe. In particular, in some embodiments, the act 830 includes determining, utilizing a probe-classification-machine-learning model, a favorable probe accuracy class for the first oligonucleotide probe based on the first nucleotide sequence and an unfavorable probe accuracy class for the second oligonucleotide probe based on the second nucleotide sequence. In some cases, the probe-classification-machine-learning model comprises a neural network or one or more decision trees.

Further, in some cases, determining the favorable probe accuracy class or the unfavorable probe accuracy class comprises determining a score indicating a genotyping probability that the first oligonucleotide probe or the second oligonucleotide probe yields an accurate genotype call or a binding probability that the first oligonucleotide probe or the second oligonucleotide probe accurately binds to a target oligonucleotide for genotyping.

In addition to the acts 810-820, in some embodiments, the acts 800 include selecting the first oligonucleotide probe for use in a microarray based on the favorable probe accuracy class for the first oligonucleotide probe; and presenting, for display within a graphical user interface of the computing device, a representation of the first oligonucleotide probe as part of a recommended set of oligonucleotide probes for use in the microarray.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleobase type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using 7-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the microarray system 104 or the probe design system 106 can include software, hardware, or both. For example, the components of the microarray system 104 can include one or more instructions stored on a computer-readable storage medium and executable by processors of one or more computing devices (e.g., the user client device 110). When executed by the one or more processors, the computer-executable instructions of the microarray system 104 can cause the computing devices to perform the bubble detection methods described herein. Alternatively, the components of the microarray system 104 can comprise hardware, such as special purpose processing devices to perform a certain function or group of functions. Additionally, or alternatively, the components of the microarray system 104 can include a combination of computer-executable instructions and hardware.

Furthermore, the components of the microarray system 104 performing the functions described herein with respect to the microarray system 104 may, for example, be implemented as part of a stand-alone application, as a module of an application, as a plug-in for applications, as a library function or functions that may be called by other applications, and/or as a cloud-computing model. Thus, components of the microarray system 104 may be implemented as part of a stand-alone application on a personal computing device or a mobile device. Additionally, or alternatively, the components of the microarray system 104 may be implemented in any application that provides sequencing services including, but not limited to Illumina Infinium, Illumina BeadChips, Infinium Global Screening Array, or Infinium Global Diversity Array. “Illumina,” “Infinium,” “BeadChips,” “Global Screening Array,” and “Global Diversity Array,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 9 illustrates a block diagram of a computing device 900 that may be configured to perform one or more of the processes described above. One will appreciate that one or more computing devices such as the computing device 900 may implement the probe design system 106 and the microarray system 104. As shown by FIG. 9, the computing device 900 can comprise a processor 902, a memory 904, a storage device 906, an I/O interface 908, and a communication interface 910, which may be communicatively coupled by way of a communication infrastructure 912. In certain embodiments, the computing device 900 can include fewer or more components than those shown in FIG. 9. The following paragraphs describe components of the computing device 900 shown in FIG. 9 in additional detail.

In one or more embodiments, the processor 902 includes hardware for executing instructions, such as those making up a computer program. As an example, and not by way of limitation, to execute instructions for dynamically modifying workflows, the processor 902 may retrieve (or fetch) the instructions from an internal register, an internal cache, the memory 904, or the storage device 906 and decode and execute them. The memory 904 may be a volatile or non-volatile memory used for storing data, metadata, and programs for execution by the processor(s). The storage device 906 includes storage, such as a hard disk, flash disk drive, or other digital storage device, for storing data or instructions for performing the methods described herein.

The I/O interface 908 allows a user to provide input to, receive output from, and otherwise transfer data to and receive data from computing device 900. The I/O interface 908 may include a mouse, a keypad or a keyboard, a touch screen, a camera, an optical scanner, network interface, modem, other known I/O devices or a combination of such I/O interfaces. The I/O interface 908 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 908 is configured to provide graphical data to a display for presentation to a user. The graphical data may be representative of one or more graphical user interfaces and/or any other graphical content as may serve a particular implementation.

The communication interface 910 can include hardware, software, or both. In any event, the communication interface 910 can provide one or more interfaces for communication (such as, for example, packet-based communication) between the computing device 900 and one or more other computing devices or networks. As an example, and not by way of limitation, the communication interface 910 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 910 may facilitate communications with various types of wired or wireless networks. The communication interface 910 may also facilitate communications using various communication protocols. The communication infrastructure 912 may also include hardware, software, or both that couples components of the computing device 900 to each other. For example, the communication interface 910 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A method comprising:
identifying candidate oligonucleotide probes for hybridizing with target oligonucleotides;
determining a nucleotide sequence of an oligonucleotide probe from the candidate oligonucleotide probes; and
determining, utilizing a probe-classification-machine-learning model, a probe accuracy classification for the oligonucleotide probe based on the nucleotide sequence of the oligonucleotide probe.

2. The method of claim 1, wherein determining the probe accuracy classification comprises determining, for the oligonucleotide probe, a favorable genotyping accuracy class indicating a probability that the oligonucleotide probe yields an accurate genotype call or an unfavorable genotyping accuracy class indicating a probability that the oligonucleotide probe yields an inaccurate genotype call.

3. The method of claim 1, wherein determining the probe accuracy classification comprises determining, for the oligonucleotide probe, a favorable binding accuracy class indicating a probability that the oligonucleotide probe accurately binds to a target oligonucleotide for genotyping or an unfavorable binding accuracy class indicating a probability that the oligonucleotide probe inaccurately binds to the target oligonucleotide for genotyping.

4. The method of claim 1, wherein determining the probe accuracy classification comprises determining a score indicating a genotyping probability that the oligonucleotide probe yields an accurate genotype call or a binding probability that the oligonucleotide probe accurately binds to a target oligonucleotide for genotyping.

5. The method of claim 1, further comprising selecting the oligonucleotide probe for use in a microarray based on the probe accuracy classification.

6. The method of claim 1, further comprising:
hybridizing, utilizing a microarray, one or more copies of the oligonucleotide probe with one or more copies of a target oligonucleotide from a genomic sample; and
determining a variant call for the genomic sample based on one or more copies of the oligonucleotide probe hybridizing with one or more copies of the target oligonucleotide.

7. The method of claim 1, further comprising:
determining, by the probe-classification-machine-learning model, feature values representing a pattern within the nucleotide sequence of the oligonucleotide probe corresponding to complimentary nucleobase bonds between the oligonucleotide probe and a target oligonucleotide; and
determining, by the probe-classification-machine-learning model, the probe accuracy classification for the oligonucleotide probe based on the feature values representing the pattern.

8. The method of claim 7, wherein determining the feature values representing the pattern within the nucleotide sequence comprises utilizing one or more filters of a kernel size customized for nucleotide-sequence-pattern recognition to determine the feature values representing the pattern.

9. The method of claim 1, further comprising:
determining, by the probe-classification-machine-learning model, feature values corresponding to nucleobases of one or more nucleobase classes within the nucleotide sequence of the oligonucleotide probe utilizing one or more channels customized for nucleobase-class recognition; and
determining the probe accuracy classification for the oligonucleotide probe based on the feature values corresponding to the nucleobases of one or more nucleobase classes.

10. A system comprising:
at least one processor; and
a non-transitory computer readable medium comprising instructions that, when executed by the at least one processor, cause the system to:
identify candidate oligonucleotide probes for hybridizing with target oligonucleotides;
determine a nucleotide sequence of an oligonucleotide probe from the candidate oligonucleotide probes; and
determine, utilizing a probe-classification-machine-learning model, a probe accuracy classification for the oligonucleotide probe based on the nucleotide sequence of the oligonucleotide probe.

11. The system of claim 10, further comprising instructions that, when executed by the at least one processor, cause the system to:
determine a different nucleotide sequence of an additional oligonucleotide probe from the candidate oligonucleotide probes; and
determine, utilizing the probe-classification-machine-learning model, a different probe accuracy classification for the additional oligonucleotide probe based on the different nucleotide sequence of the additional oligonucleotide probe.

12. The system of claim 10, further comprising instructions that, when executed by the at least one processor, cause the system to:
identify threshold ranges for genotyping metrics indicating accurate probes and inaccurate probes for genotyping; and
categorize, based on the threshold ranges for genotyping metrics, the candidate oligonucleotide probes into a favorable probe-accuracy-training class for training the probe-classification-machine-learning model and an unfavorable probe-accuracy-training class for training the probe-classification-machine-learning model.

13. The system of claim 12, further comprising instructions that, when executed by the at least one processor, cause the system to:
identify, from among the favorable probe-accuracy-training class or the unfavorable probe-accuracy-training class, a ground-truth oligonucleotide probe corresponding to the oligonucleotide probe;
determine a value difference between a ground-truth probe accuracy classification for the ground-truth oligonucleotide probe and the probe accuracy classification for the oligonucleotide probe; and
modify one or more network parameters of the probe-classification-machine-learning model based on the value difference.

14. The system of claim 10, further comprising instructions that, when executed by the at least one processor, cause the system to determine the probe accuracy classification by determining, for the oligonucleotide probe and based on a dataset representing the nucleotide sequence, a favorable genotyping accuracy class indicating a probability that the oligonucleotide probe yields an accurate genotype call or an unfavorable genotyping accuracy class indicating a probability that the oligonucleotide probe yields an inaccurate genotype call.

15. The system of claim 10, further comprising instructions that, when executed by the at least one processor, cause the system to determine the probe accuracy classification by determining, for the oligonucleotide probe and based on a dataset representing the nucleotide sequence, a favorable binding accuracy class indicating a probability that the oligonucleotide probe accurately binds to a target oligonucleotide for genotyping or an unfavorable binding accuracy class indicating a probability that the oligonucleotide probe inaccurately binds to the target oligonucleotide for genotyping.

16. The system of claim 10, further comprising instructions that, when executed by the at least one processor, cause the system to:
hybridize, utilizing a microarray, one or more copies of the oligonucleotide probe with one or more copies of a target oligonucleotide corresponding to one or more genomic coordinates for a promoter region or a gene from a genomic sample; and
determine a variant call for the one or more genomic coordinates of the genomic sample based on one or more copies of the oligonucleotide probe hybridizing with one or more copies of the target oligonucleotide.

17. A non-transitory computer readable medium comprising instructions that, when executed by at least one processor, cause a computing device to:
identify candidate oligonucleotide probes for hybridizing with target oligonucleotides;
determine a first nucleotide sequence of a first oligonucleotide probe from the candidate oligonucleotide probes and a second nucleotide sequence of a second oligonucleotide probe from the candidate oligonucleotide probes; and
determine, utilizing a probe-classification-machine-learning model, a favorable probe accuracy class for the first oligonucleotide probe based on the first nucleotide sequence and an unfavorable probe accuracy class for the second oligonucleotide probe based on the second nucleotide sequence.

18. The non-transitory computer readable medium of claim 17, wherein the probe-classification-machine-learning model comprises a neural network or one or more decision trees.

19. The non-transitory computer readable medium of claim 17, further comprising instructions that, when executed by at least one processor, cause the computing device to determine the favorable probe accuracy class or the unfavorable probe accuracy class by determining a score indicating a genotyping probability that the first oligonucleotide probe or the second oligonucleotide probe yields an accurate genotype call or a binding probability that the first oligonucleotide probe or the second oligonucleotide probe accurately binds to a target oligonucleotide for genotyping.

20. The non-transitory computer readable medium of claim 17, further comprising instructions that, when executed by at least one processor, cause the computing device to:
select the first oligonucleotide probe for use in a microarray based on the favorable probe accuracy class for the first oligonucleotide probe; and
present, for display within a graphical user interface of the computing device, a representation of the first oligonucleotide probe as part of a recommended set of oligonucleotide probes for use in the microarray.
